(19) United States
(12) Patent Application Publication
(10) Pub. No.: US 2008/0261974 A1
(43) Pub. Date: Oct. 23, 2008

(54) Novel Chemical Compounds

(76) Inventors: Kevin J Duffy, Collegevile, PA (US); Duke M. Fitch, Colleville, PA (US); Rosanna Tedesco, Collegeville, PA (US); Michael N. Zimmerman, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/067,471

(22) PCT Filed: Sep. 22, 2006
(86) PCT No.: PCT/US06/37090

§ 371 Date: Mar. 20, 2008

Related U.S. Application Data
(60) Provisional application No. 60/719,841, filed on Sep. 23, 2005.

(51) Int. Cl. A61K 31/5377; A61P 7/06; A61P 7/00; C07D 417/02; C07D 417/14; A61K 31/502; A61K 31/4985; A61K 31/4725; A61K 31/498; C07D 487/04; C07D 417/06; A61K 31/53; A61K 31/517

(52) U.S. Cl. 514/234.5; 514/266.24; 544/283; 544/119; 544/235; 544/346; 546/146; 544/353; 544/183; 514/243; 514/248; 514/250; 514/314; 514/249

(57) ABSTRACT

This invention relates to newly identified compounds for inhibiting hYAK3 proteins and methods for treating diseases associated with the imbalance or inappropriate activity of hYAK3 proteins.

### FIELD OF THE INVENTION

This invention relates to newly identified compounds for inhibiting hYAK3 proteins and methods for treating diseases associated with hYAK3 activity.

### BACKGROUND OF THE INVENTION

A number of polypeptide growth factors and hormones mediate their cellular effects through a signal transduction pathway. Transduction of signals from the cell surface receptors for these ligands to intracellular effectors frequently involves phosphorylation or dephosphorylation of specific protein substrates by regulatory protein serine/threonine kinases (PSTK) and phosphatases. Serine/threonine phosphorylation is a major mediator of signal transduction in multicellular organisms. Receptor-bound, membrane-bound and intracellular PSTKs regulate cell proliferation, cell differentiation and signalling processes in many cell types.

Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are potential targets for drug design.

A subset of PSTKs are involved in regulation of cell cycling. These are the cyclin-dependent kinases or CDKs (Peter and Herskowitz, Cell 1994: 79, 181-184). CDKs are activated by binding to regulatory proteins called cyclins and control passage of the cell through specific cell cycle checkpoints. For example, CDK2 complexed with cyclin E allows cells to progress through the G1 to S phase transition. The complexes of CDKs and cyclins are subject to inhibition by low molecular weight proteins such as p16 (Serrano et al, Nature 1993: 366, 704), which binds to and inhibits CDK4. Deletions or mutations in p16 have been implicated in a variety of tumors (Kamb et al, Science 1994: 264, 436-440). Therefore, the proliferative state of cells and diseases associated with this state are dependent on the activity of CDKs and their associated regulatory molecules. In diseases such as cancer where inhibition of proliferation is desired, compounds that inhibit CDKs may be useful therapeutic agents. Conversely, activators of CDKs may be useful where enhancement of proliferation is needed, such as in the treatment of immunodeficiency.

YAK1, a PSTK with sequence homology to CDKs, was originally identified in yeast as a mediator of cell cycle arrest caused by inactivation of the cAMP-dependent protein kinase PKA (Garrett et al, Mol Cell Biol. 1991: 11-6045-4052). YAK1 kinase activity is low in cycling yeast but increases dramatically when the cells are arrested prior to the S-G2 transition. Increased expression of YAK1 causes growth arrest in yeast cells deficient in PKA. Therefore, YAK1 can act as a cell cycle suppressor in yeast.

U.S. Pat. No. 6,323,318 describes two novel human homologs of yeast YAK1 termed hYAK3-2, one protein longer than the other by 20 amino acids. hYAK3-2 proteins (otherwise reported as REDK-L and REDK-S in Blood, 1 May 2000, Vol 95, No. 9, pp 2838) are primarily localized in the nucleus. hYAK-2 proteins (hereinafter simply referred as hYAK3 or hYAK3 proteins) are present in hematopoietic tissues, such as bone marrow and fetal liver, but the RNA is expressed at significant levels only in erythroid or erthropoietin (EPO)-responsive cells. Two forms of REDK cDNAs appear to be alternative splice products. Antisense REDK oligonucleotides promote erythroid colony formation by human bone marrow cells, without affecting colony-forming unit (CFU)-GM, CFU-G, or CFU-GEMM numbers. Maximal numbers of CFU-E and burst-forming unit-erythroid were increased, and CFU-E displayed increased sensitivity to suboptimal EPO concentrations. The data indicate that REDK acts as a brake to retard erythropoiesis. Thus inhibitors of hYAK3 proteins are expected to stimulate proliferation of cells in which it is expressed. More particularly, inhibitors of hYAK3 proteins are useful to treat or prevent diseases of the erythroid and hematopoietic systems associated with hYAK3 activity, including but not limited to anemia, anemias due to renal insufficiency or to chronic disease, such as autoimmunity, HIV, or cancer, and drug-induced anemias, myelodysplastic syndrome, aplastic anemia and myelosuppression, and cytopenia.

### SUMMARY OF THE INVENTION

This invention relates to a method of inhibiting hYAK3 in a mammal with compounds of Formula (I):

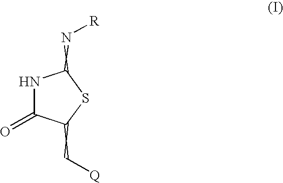

in which

R is selected form: aryl and substituted aryl; and

### Q is

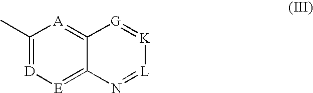

wherein
A, D and E are independently selected from CR²⁰ and N, and
G, K and L are independently selected from CR²⁰ and N,
G and K optionally form a five-membered ring containing 1-4 nitrogens,
where each R²⁰ is independently selected from the group consisting of: hydrogen, amino, alkylamino, substituted alkylamino, dialkylamino, substituted dialkylamino, hydroxy, alkylaminoalkyl, dialkylaminoalkyl, alkoxy, alkyl, substituted alkyl, aryl, substituted aryl, arylamino, substituted arylamino, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected form hydrogen, C₁-C₄alkyl, aryl and
trifluoromethyl, and R¹¹ and R¹² are independently selected from hydrogen, C₁-C₄alkyl, aryl and trifluoromethyl;
   and/or pharmaceutically acceptable salts, hydrates, solvates and pro-drugs thereof;
   provided that not each of G, K and L are N,
   further provided that when G is CR²⁰, at least one of A, D, E, K, and L is N,
   further provided that when G is N, Q must contain at least three nitrogens.

This invention relates a method of inhibiting hYAK3 in a mammal; comprising administering to the mammal a therapeutically effective amount of a compound of the formula (I).

This invention relates to a method of treating or preventing diseases of the erythroid and hematopoietic systems, caused by hYAK3 activity including, but not limited to, anemia, anemias due to renal insufficiency or to chronic disease, such as autoimmunity, HIV, or cancer, and drug-induced anemias, myelodysplastic syndrome, aplastic anemia and myelosuppression, and cytopenia; comprising administering to a mammal a therapeutically effective amount of a compound of formula (I).

In a further aspect of the invention there is provided novel processes and novel intermediates useful in preparing the presently invented hYAK3 inhibiting compounds.

Included in the present invention are pharmaceutical compositions that comprise a pharmaceutical carrier and compounds useful in the methods of the invention.

Also included in the present invention are methods of co-administering the presently invented hYAK3 inhibiting compounds with further active ingredients.

### DETAILED DESCRIPTION

This invention relates to a method of inhibiting hYAK3 with compounds of Formula (I) as described above.

This invention relates to compounds of Formula (I) as described above.

This invention relates to a method as described above,

### Wherein

A, D and E are independently selected from CR²⁰ and N, and
G, K and L are independently selected from CR²⁰ and N,
where each R²⁰ is independently selected from the group consisting of: hydrogen, amino, alkylamino, substituted alkylamino, dialkylamino, substituted dialkylamino, hydroxy, alkylaminoalkyl, dialkylaminoalkyl, alkoxy, alkyl, substituted alkyl, aryl, substituted aryl, arylamino, substituted arylamino, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected form hydrogen, C₁-C₄alkyl, aryl and
trifluoromethyl, and R¹¹ and R¹² are independently selected from hydrogen, C₁-C₄alkyl, aryl and trifluoromethyl;
   and/or pharmaceutically acceptable salts, hydrates, solvates and pro-drugs thereof;
   provided that not each of G, K and L are N,
   further provided that when G is CR²⁰, at least one of A, D, E, K, and L is N,
   further provided that when G is N, Q must contain at least three nitrogens.

This invention relates to compounds of Formula (I) as described above, wherein
A, D and E are independently selected from CR²⁰ and N, and
G, K and L are independently selected from CR²⁰ and N,
where each R²⁰ is independently selected from the group consisting of: hydrogen, amino, alkylamino, substituted alkylamino, dialkylamino, substituted dialkylamino, alkylaminoalkyl, dialkylaminoalkyl, alkyl, substituted alkyl, aryl, substituted aryl, arylamino, substituted arylamino, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected form hydrogen, C₁-C₄alkyl, aryl and
trifluoromethyl, and R¹¹ and R¹² are independently selected from hydrogen, C₁-C₄alkyl, aryl and trifluoromethyl;
   and/or pharmaceutically acceptable salts, hydrates, solvates and pro-drugs thereof;
   provided that not each of G, K and L are N,
   further provided that when G is CR²⁰, at least one of A, D, E, K, and L is N,
   further provided that when G is N, Q must contain at least three nitrogens.

This invention relates to compounds of Formula (I) as described above, wherein at least one of A, D, and E is N.

The presently invented compounds of Formula (I) inhibit hYAK3 activity.

Included among the presently invented compounds of Formula (I) are those having Formula (II):

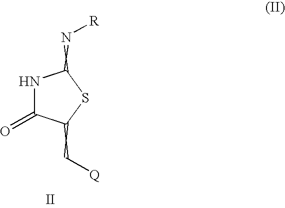

in which
R is selected form: C₁-C₁₂aryl and substituted C₁-C₁₂aryl; and
Q is naphthyridin-6-yl, substituted naphthyridin-6-yl, naphthyl, 3-isoquinolinyl, 2-quinoxalinyl, quinazolin-6-yl, substituted quinazolin-6-yl, cinnolin-6-yl, substituted cinnolin-6-yl, or a substituent of formula (IV):

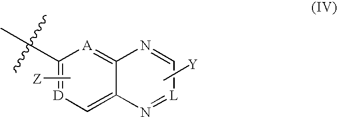

wherein
A, D and L are CR²⁰ or N,
where R²⁰, Z and Y are independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected form hydrogen, C₁-C₄alkyl, aryl and
trifluoromethyl, and R¹¹ and R¹² are independently selected from hydrogen, C₁-C₄alkyl, aryl and trifluoromethyl,
provided that at least one of A, D and L is N;
   and/or pharmaceutically acceptable salts, hydrates, solvates and pro-drugs thereof.

Included among the presently invented compounds of Formulas (I) and (II) are those in which:

### R is

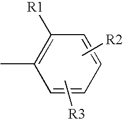 [0047] in which R¹ is hydrogen, halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, —OCF₃, or —CO₂C₁₋₆alkyl; and
R² and R³ are independently hydrogen, halogen, —C₁₋₆ alkyl, substituted —C₁₋₆alkyl, C₁-C₁₂aryl, cycloalkyl, cycloalkyl containing from 1 to 4 heteroatoms, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, —CO₂C₁₋₆alkyl, —NH₂, alkylamino, dialkylamino, —OCH₂(C═O)OH, —OCH₂CH₂OCH₃, —SO₂NH₂,
—S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—NR⁴¹C(O)R³¹, where R³¹ is selected from aryl, -Oalkyl, -Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl,
and R⁴¹ is selected from hydrogen and C₁-C₆alkyl,
—NR⁴⁴S(O)₂R³⁴, where R³⁴ is selected from hydrogen, alkyl, cycloalkyl, substituted cycloalkyl, cycloalkyl containing 1 to 4 heteroatoms,
substituted cycloalkyl containing 1 to 4 heteroatoms and aryl, and R⁴⁴ is selected from hydrogen and C₁-C₆alkyl,
—CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl, —CO₂C₁₋₆alkyl, —NH₂, alkylamino, dialkylamino or —NH(C═NH)CH₃; and
Q is naphthyl, 3-isoquinolinyl, 2-quinoxalinyl, naphthyridin-6-yl, substituted naphthyridin-6-yl, quinazolin-6-yl, substituted quinazolin-6-yl, cinnolin-6-yl, substituted cinnolin-6-yl, or a substituent of formula (IV):

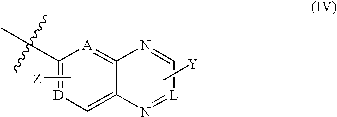

wherein
A, D and L are CR²⁰ or N,
where R²⁰, Z and Y are independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected form hydrogen, C₁-C₄alkyl, aryl and
trifluoromethyl, and R¹¹ and R¹² are independently selected from hydrogen, C₁-C₄alkyl, aryl and trifluoromethyl,
provided that at least one of A, D and L is N;
   and/or pharmaceutically acceptable salts, hydrates, solvates and pro-drugs thereof.

Included among the presently invented compounds of Formulas (I) and (II) are those in which R is:

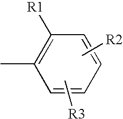

in which R1 is halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, or —CO₂C₁₋₆alkyl; and
R2 and R3 are independently hydrogen, halogen, —C₁₋₆ alkyl, substituted —C₁₋₆ alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, —S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—NR⁴¹C(O)R³¹, where R³¹ is selected from aryl, -Oalkyl, -Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁-C₆alkyl,
—NR⁴⁴S(O)₂R³⁴, where R³⁴ is selected from hydrogen, alkyl, cycloalkyl, substituted cycloalkyl, cycloalkyl containing 1 to 4 heteroatoms,
substituted cycloalkyl containing 1 to 4 heteroatoms and aryl, and R⁴⁴ is selected from hydrogen and C₁-C₆alkyl,
—CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl,
—CO₂C₁₋₆alkyl, —NH₂, alkylamino, dialkylamino or —NH(C═NH)CH₃;
and
Q is naphthyl, 3-isoquinolinyl, 2-quinoxalinyl, naphthyridin-6-yl, substituted naphthyridin-6-yl, quinazolin-6-yl, substituted quinazolin-6-yl, cinnolin-6-yl, substituted cinnolin-6-yl, or a substituent of formula (IV):

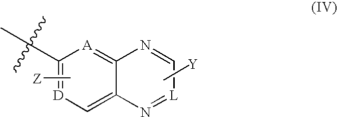

wherein
A, D and L are CR²⁰ or N,
where R²⁰, Z and Y are independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected form hydrogen, C₁-C₄alkyl, aryl and
trifluoromethyl, and R¹¹ and R¹² are independently selected from hydrogen, C₁-C₄alkyl, aryl and trifluoromethyl,
provided that at least one of A, D and L is N;
   and/or pharmaceutically acceptable salts, hydrates, solvates and pro-drugs thereof.

Included among the presently invented compounds of Formulas (I) and (II) are those in which:

### R is

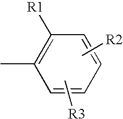 [0078] in which R¹ is halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, or —CO₂C₁₋₆alkyl;
and
R2 and R3 are independently hydrogen, halogen, —C₁₋₆ alkyl, substituted —C₁₋₆ alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H,
—S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—NR⁴¹C(O)R³¹, where R³¹ is selected from aryl, -Oalkyl, -Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁-C₆alkyl,
—NR⁴⁴S(O)₂R³⁴, where R³⁴ is selected from hydrogen, alkyl, cycloalkyl, substituted cycloalkyl, cycloalkyl containing 1 to 4 heteroatoms, substituted cycloalkyl containing 1 to 4 heteroatoms and aryl, and R⁴⁴ selected from hydrogen and C₁-C₆alkyl,
—CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl,
—CO₂C₁₋₆alkyl, —NH₂, alkylamino or —NH(C═NH)CH₃;
and
Q is naphthyl, 3-isoquinolinyl, 2-quinoxalinyl, naphthyridin-6-yl, substituted naphthyridin-6-yl, quinazolin-6-yl, substituted quinazolin-6-yl, cinnolin-6-yl, substituted cinnolin-6-yl, or a substituent of formula (IV):

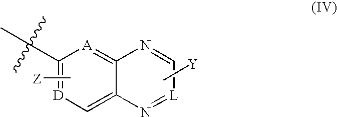

wherein
A, D and L are CR²⁰ or N,
where R²⁰, Z and Y are independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected form hydrogen, C₁-C₄alkyl, aryl and
trifluoromethyl, and R¹¹ and R¹² are independently selected from hydrogen, C₁-C₄alkyl, aryl and trifluoromethyl,

provided that at least one of A, D and L is N;

and/or pharmaceutically acceptable salts, hydrates, solvates and pro-drugs thereof.

Included among the presently invented compounds of Formulas (I) and (II) are those in which:

### R is

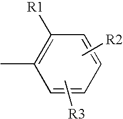 [0095] in which R1 is halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, or —CO₂C₁₋₆alkyl;
and
R2 and R3 are independently hydrogen, halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —OH, —CF₃, —CN, —CO₂H,
—S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl, —NR⁴¹C(O)R³¹, where R³¹ is selected from aryl, -Oalkyl, -Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁-C₆alkyl,
—NR⁴⁴S(O)₂R³⁴, where R³⁴ is selected from hydrogen, alkyl, cycloalkyl, substituted cycloalkyl, cycloalkyl containing 1 to 4 heteroatoms, substituted cycloalkyl containing 1 to 4 heteroatoms and aryl, and R⁴⁴ is selected from hydrogen and C₁-C₆alkyl, —CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl,
—CO₂C₁₋₆alkyl, —NH₂, alkylamino, or —NH(C═NH)CH₃;
and
Q is naphthyl, 3-isoquinolinyl, 2-quinoxalinyl, naphthyridin-6-yl, substituted naphthyridin-6-yl, quinazolin-6-yl, substituted quinazolin-6-yl, cinnolin-6-yl, substituted cinnolin-6-yl, or a substituent of formula (IV):

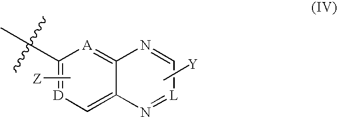

wherein
A, D and L are CR²⁰ or N,
where R²⁰, Z and Y are independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected form hydrogen, C₁-C₄alkyl, aryl and
trifluoromethyl, and R¹¹ and R¹² are independently selected from hydrogen, C₁-C₄alkyl, aryl and trifluoromethyl,
provided that at least one of A, D and L is N;
   and/or pharmaceutically acceptable salts, hydrates, solvates and pro-drugs thereof.

Also Included among the presently invented compounds of Formula (I) are those in which R is C₁-C₁₂aryl or substituted C₁-C₁₂aryl, A, D, and E are CR²⁰, G, K and L are selected from CR²⁰ and N, where R²⁰ is selected from the group consisting of: hydrogen, amino, alkylamino, substituted alkylamino, dialkylamino, substituted dialkylamino, hydroxy, alkylaminoalkyl, dialkylaminoalkyl, alkoxy, alkyl, substituted alkyl, aryl, substituted aryl, arylamino, substituted arylamino, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,

where, R¹⁰ is selected from hydrogen, C₁-C₄alkyl, aryl and trifluoromethyl, and R¹¹ and R¹² are each independently selected from hydrogen, C₁-C₄alkyl, aryl and trifluoromethyl;

and/or a pharmaceutically acceptable salt, hydrate, solvate or pro-drug thereof;

provided that when G is N, G and K together form a fused five-membered ring containing 1-4 nitrogens.

Also Included among the presently invented compounds of Formula (I) are those in which R is C₁-C₁₂aryl or substituted C₁-C₁₂aryl, A, D, L and E are CH, G, K together form a five-membered ring containing one to four nitrogens; and/or a pharmaceutically acceptable salt, hydrate, solvate or pro-drug thereof.

Also included among the presently invented compound are those of formula

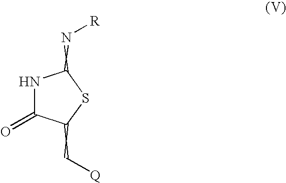

wherein R is C₁-C₁₂aryl or substituted C₁-C₁₂aryl,

Q is a selected from a group consisting of: formula VI, VII, VIII

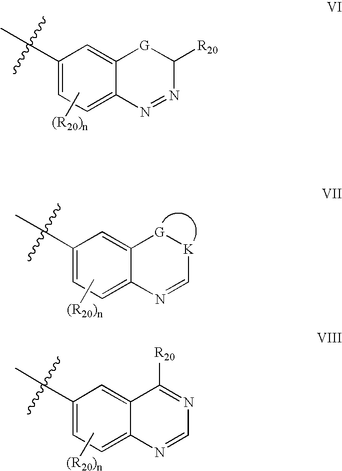

where n is 0-3, G and K are each independently selected from N or CR²⁰,
G and K optionally form a five-membered ring containing 1-4 nitrogens, each R²⁰ is independently selected from the group consisting of: hydrogen, amino, alkylamino, substituted alkylamino, dialkylamino, substituted dialkylamino, alkylaminoalkyl, dialkylaminoalkyl, alkyl, substituted alkyl, aryl, substituted aryl, arylamino, substituted arylamino, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected from hydrogen, C₁-C₄alkyl, aryl and
trifluoromethyl, and R¹¹ and R¹² are each independently selected from hydrogen, C₁-C₄alkyl, aryl and trifluoromethyl;
   and/or a pharmaceutically acceptable salt, hydrate, solvate or pro-drug thereof;

provided that when Q is formula VIII, R²⁰ is not a hydrogen,

Also included among the novel compounds of current invention are compounds of formula V wherein Q is formula VI, and/or a pharmaceutically acceptable salt, hydrate, solvate or pro-drug thereof.

Also included among the novel compounds of current invention are compounds of formula V wherein Q is formula VII, and/or a pharmaceutically acceptable salt, hydrate, solvate or pro-drug thereof.

Also included among the novel compounds of current invention are compounds of formula V wherein Q is formula VIII, and R²⁰ is selected from a group consisting of: amino, alkylamino, dialkylamino, substituted alkylamino, arylamino, oxo, and substituted arylamino; and/or a pharmaceutically acceptable salt, hydrate, solvate or pro-drug thereof.

Included among the novel compounds useful in the present invention are:
(5Z)-2-[(2,6-Dichlorophenyl)amino]-5-(6-quinazolinylmethylidene)-1,3-thiazol-4(5H)-one;
(5Z)-2-[(2,6-Dichlorophenyl)amino]-5-{[4-(4-morpholinyl)-6-quinazolinyl]methylidene}-1,3-thiazol-4(5H)-one;
(5Z)-5-(6-Cinnolinylmethylidene)-2-[(2,6-dichlorophenyl)amino]-1,3-thiazol-4(5H)-one;
(5Z)-2-[(2,6-Dichlorophenyl)amino]-5-(imidazo[1,2-a]quinoxalin-8-ylmethylidene)-1,3-thiazol-4(5H)-one;
N-(4-Chloro-3-{[(5Z)-5-(imidazo[1,2-a]quinoxalin-8-ylmethylidene)-4-oxo-4,5-dihydro-1,3-thiazol-2-yl]amino}phenyl)cyclobutanecarboxamide;
(5Z)-2-[(2,6-Dichlorophenyl)amino]-5-(tetrazolo[1,5-a]quinoxalin-8-ylmethylidene)-1,3-thiazol-4(5H)-one;
(5Z)-5-[(4-Amino-6-quinazolinyl)methylidene]-2-[(2,6-dichlorophenyl)amino]-1,3-thiazol-4(5H)-one trifluoroacetate;
(5Z)-2-[(2,6-Dichlorophenyl)amino]-5-{[4-(methylamino)-6-quinazolinyl]methylidene}-1,3-thiazol-4(5H)-one trifluoroacetate;
6-{(Z)-[2-[(2,6-Dichlorophenyl)amino]-4-oxo-1,3-thiazol-5(4H)-ylidene]methyl}-4(1H)-quinazolinone, piperidine salt;
(5Z)-2-[(2,6-Dichlorophenyl)amino]-5-{[4-(dimethylamino)-6-quinazolinyl]methylidene}-1,3-thiazol-4(5H)-one;
(5Z)-5-{[4-(Methylamino)-6-quinazolinyl]methylidene}-2-[(2,4,6-trichlorophenyl)amino]-1,3-thiazol-4(5H)-one hydrochloride;
4-[(6-{(Z)-[2-[(2,6-Dichlorophenyl)amino]-4-oxo-1,3-thiazol-5(4H)-ylidene]methyl}-4-quinazolinyl)amino]-N,N-dimethylbenzenesulfonamide;
(5Z)-2-[(2,6-Dichlorophenyl)amino]-5-(3-isoquinolinylmethylidene)-1,3-thiazol-4(5H)-one;
(5Z)-2-[(2,6-Dichlorophenyl)amino]-5-(2-quinoxalinylmethylidene)-1,3-thiazol-4(5H)-one;
Ethyl6-{(Z)-[2-[(2,6-dichlorophenyl)amino]-4-oxo-1,3-thiazol-5(4H)-ylidene]methyl}-1,2,4-benzotriazine-3-carboxylate; and
(5Z)-2-[(2,6-Dichlorophenyl)amino]-5-(2-naphthalenylmethylidene)-1,3-thiazol-4(5H)-one.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compounds of Formula (I) are included in the pharmaceutical compositions of the invention and used in the methods of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, 3,4-methylenedioxyphenyl, pyridine, biphenyl, quinoline, pyrimidine, quinazoline, thiophene, thiazole, furan, pyrrole, pyrazole, imidazole, indole, indene, pyrazine, 1,3-dihydro-2H-benzimidazol, benzimidazol, benzothiophene, tetrahydrobenzothiophene and tetrazole.

The term “substituted” as used herein, unless otherwise defined, is meant that the subject chemical moiety has one or more substituents selected from the group consisting of: alkyl, cycloalkyl, cycloalkyl containing from 1 to 4 heteroatoms, acyloxy, aryloxy, hydroxy, alkoxy, oxo, cyano, amino, alkylamino, dialkylamino, trifluoromethyl, —SO₂NR⁶¹R⁶², —N-acylamino, —CO₂R⁶⁰, —NC(O)R⁷⁰, halogen, aryl, aryl substituted with one to five substituents selected from alkyl, C₁-C₆cycloalkyl, hydroxy, alkoxy, oxo, cyano, amino, alkylamino, dialkylamino, trifluoromethyl, —SO₂NR⁶¹R⁶², N-acylamino, —CO₂R⁶⁰, —NC(O)R⁷⁰, and halogen, where R⁶¹, R⁶², R⁶⁰ and R⁷⁰ are hydrogen, cycloalkyl or C1-C4alkyl.

By the term “naphthyridin-6-yl” as used herein, is meant 1,5-naphthyridin-6-yl, 1,7-naphthyridin-6-yl, and 1,8-naphthyridin-6-yl.

By the term “alkoxy” as used herein is meant -Oalkyl where alkyl is as described herein including —OCH₃ and —OC(CH₃)₂CH₃.

The term “cycloalkyl” as used herein unless otherwise defined, is meant a nonaromatic, unsaturated or saturated, cyclic or polycyclic C₃-C₁₂.

Examples of cycloalkyl and substituted cycloalkyl substituents as used herein include: cyclohexyl, aminocyclohexyl, cyclobutyl, aminocyclobutyl, 4-hydroxy-cyclohexyl, 2-ethylcyclohexyl, propyl-4-methoxycyclohexyl, 4-methoxycyclohexyl, 4-carboxycyclohexyl, cyclopropyl, aminocyclopentyl, and cyclopentyl.

The term “cycloalkyl containing from 1 to 4 heteroatoms” and the term “cycloalkyl containing from 1 to 3 heteroatoms” as used herein unless otherwise defined, is meant a nonaromatic, unsaturated or saturated, cyclic or polycyclic ring containing from 1 to 12 carbons and containing from one to four heteroatoms or from one to three heteroatoms (respectively), provided that when the number of carbon atoms is 1 the aromatic ring contains at least four heteroatoms (applicable only where “cycloalkyl containing from 1 to 4 heteroatoms” is indicated), when the number of carbon atoms is 2 the aromatic ring contains at least three heteroatoms, when the number of carbon atoms is 3 the nonaromatic ring contains at least two heteroatoms and when the number of carbon atoms is 4 the nonaromatic ring contains at least one heteroatom.

Examples of cycloalkyl containing from 1 to 4 heteroatoms, cycloalkyl containing from 1 to 3 heteroatoms, substituted cycloalkyl containing from 1 to 4 heteroatoms and substituted cycloalkyl containing from 1 to 3 heteroatoms as used herein include: piperidine, piperazine, pyrrolidine, 3-methylaminopyrrolidine, piperazine, tetrazole, hexahydrodiazepine and morpholine.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant -Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifluoromethyl, acyloxy, amino, N-acylamino, hydroxy, —(CH₂)_{g}C(O)OR⁶⁵, —S(O)ₙR⁶⁵, nitro, cyano, halogen and protected —OH, where g is 0-6, R²⁵ is hydrogen or alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein, including alkyl chains defined by the term “—(CH₂)ₙ”, “—(CH₂)ₘ” and the like, is meant a linear or branched, saturated or unsaturated hydrocarbon chain, and unless otherwise defined, the carbon chain will contain from 1 to 12 carbon atoms.

Examples of alkyl and substituted alkyl substituents as used herein include:

—CH₃, —CH₂—CH₃, —CH₂—CH₂—CH₃, —CH(CH₃)₂, —CH₂—CH₂—C(CH₃)₃, —CH₂—CF₃, —C≡C—C(CH₃)₃, —C≡C—CH₂—OH, cyclopropylmethyl, —CH₂—C(CH₃)₂—CH₂—NH₂, —C≡C—C₆H₅, —C≡C—C(CH₃)₂—OH, —CH₂—CH(OH)—CH(OH)—CH(OH)—CH(OH)—CH₂—OH, piperidinylmethyl, methoxyphenylethyl, —C(CH₃)₃, —(CH₂)₃—CH₃, —CH₂—CH(CH₃)₂, —CH(CH₃)—CH₂—CH₃, —CH═CH₂, and —C≡C—CH₃.

By the term “treating” and derivatives thereof as used herein, is meant prophylatic and therapeutic therapy.

As used herein, the term “optionally” means that the subsequently described event(s) may or may not occur, and includes both event(s), which occur, and events that do not occur.

As used herein, the crisscrossed double bond indicated by the symbol [symbol] denotes Z and/or E stereochemistry around the double bond. In other words a compound of Formula I can be either in the Z or E stereochemistry around this double bond, or a compound of Formula I can also be in a mixture of Z and E stereochemistry around the double bond. However, in formulas I and II, the preferred compounds have Z stereochemistry around the double bond to which radical Q is attached.

A person of ordinary skill can readily appreciate that certain substituents may cause the double bonds of Q in Formula I or V to shift within the bicyclic ring. An example of such substituent is: oxo.

The compounds of Formulas I and II naturally may exist in one tautomeric form or in a mixture of tautomeric forms. For example, for sake simplicity, compounds of formula I and II are expressed in one tautomeric form, usually as an exo form, i.e.

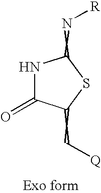

However, a person of ordinary skill can readily appreciate, the compounds of formulas I and II can also exist in endo forms.

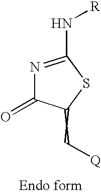

The present invention contemplates all possible tautomeric forms.

Certain compounds described herein may contain one or more chiral atoms, or may otherwise be capable of existing as two enantiomers, or two or more diastereoisomers. Accordingly, the compounds of this invention include mixtures of enantiomers/diastereoisomers as well as purified enantiomers/diastereoisomers or enantiomerically/diastereoisomerically enriched mixtures. Also included within the scope of the invention are the individual isomers of the compounds represented by Formula I above as well as any wholly or partially equilibrated mixtures thereof. The present invention also covers the individual isomers of the compounds represented by the formulas above as mixtures with isomers thereof in which one or more chiral centers are inverted. Further, an example of a possible tautomer is an oxo substituent in place of a hydroxy substituent. Also, as stated above, it is understood that all tautomers and mixtures of tautomers are included within the scope of the compounds of Formula I.

Compounds of Formula (I) are included in the pharmaceutical compositions of the invention and used in the methods of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

The novel compounds of Formulas I and II are prepared as shown in Schemes I and II below, or by analogous methods, wherein the ‘Q’ and ‘R’ substituents are as defined in Formulas I and II respectively and provided that the ‘Q’ and ‘R’ substituents do not include any such substituents that render inoperative the processes of Schemes I to II. All of the starting materials are commercially available or are readily made from commercially available starting materials by those of skill in the art.

### General Schemes

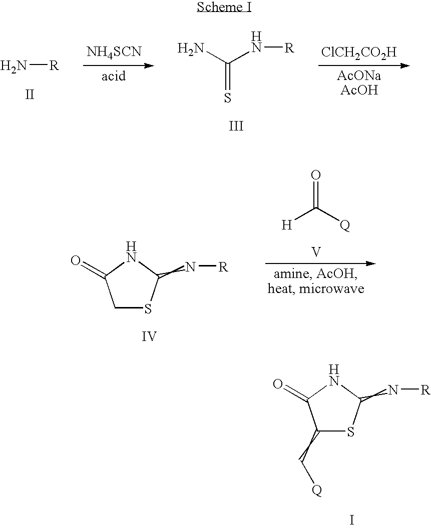

Briefly in Scheme 1, a mixture of aniline derivative of formula II (1 equivalent) and NH4SCN (about 1.3 equivalent) in an acid (typically 4N—HCl) is heated to reflux at about 110° C. for 6 hours. After cooling, the mixture is treated with H₂O, which process usually forms a solid, followed by desiccation in vacuo to give a compound of formula III.

A mixture of formula III compound, ClCH₂CO₂H (1 equivalent), and AcONa (1 equivalent) in AcOH is heated to reflux at around 110C.° for about 4 h. The mixture is poured onto water thereby a solid is typically formed, which is isolated by filtration. The solid is washed with a solvent such as MeOH to afford a compound of formula IV.

A mixture of formula IV compound, an aldehyde of formula V (1 equivalent), an amine such as piperidine, and optionally acetic acid in AcOH is heated in a microwave reactor at about 150° C. for about 0.5 hours. After cooling, a small portion of water is added until the solid forms. The solid is filtered and washed with a solvent such as MeOH, followed by desiccation in vacuo to afford a target product of Formula I.

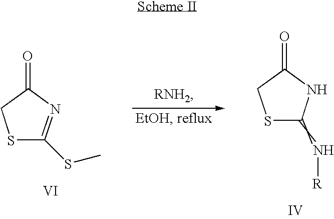

Scheme 2 shows an alternative synthesis of the intermediate IV. Briefly in Scheme 2, a mixture of the known thiazolinone VI and aniline derivative RNH₂ in ethanol is heated under reflux to give the intermediate IV after appropriate work-up.

In Schemes 1 and 2, the meaning of R and Q are as defined in Formula I.

In other embodiments, additional compounds of the invention can also be synthesized whereby a compound of Formula I is first made by a process of Scheme 1 or 2 (or a variant thereof), and Q and R radicals in compounds of Formula I thus made are further converted by routine organic reaction techniques into different Q and R groups.

By the term “co-administering” and derivatives thereof as used herein is meant either simultaneous administration or any manner of separate sequential administration of a hYAK3 inhibiting compound, as described herein, and a further active ingredient or ingredients, known to be useful in treating diseases of the hematopoietic system, particularly anemias, including EPO or a derivative thereof. The term further active ingredient or ingredients, as used herein, includes any compound or therapeutic agent known to or that demonstrates advantageous properties when administered to a patient in need of treatment for diseases of the hematopoietic system, particularly anemias. Preferably, if the administration is not simultaneous, the compounds are administered in a close time proximity to each other. Furthermore, it does not matter if the compounds are administered in the same dosage form, e.g. one compound may be administered topically and another compound may be administered orally.

Because the pharmaceutically active compounds of the present invention are active as hYAK3 inhibitors they exhibit therapeutic utility in treating diseases of the hematopoietic system, particularly anemias.

The pharmaceutically active compounds within the scope of this invention are useful as hYAK inhibitors in mammals, particularly humans, in need thereof.

The present invention therefore provides a method of treating diseases of the hematopoietic system, particularly anemias and other conditions requiring hYAK inhibition, which comprises administering an effective compound of Formula (I) or a pharmaceutically acceptable salt, hydrate, solvate or pro-drug thereof. The compounds of Formula (I) also provide for a method of treating the above indicated disease states because of their ability to act as hYAK inhibitors. The drug may be administered to a patient in need thereof by any conventional route of administration, including, but not limited to, intravenous, intramuscular, oral, subcutaneous, intradermal, and parenteral.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of a hYAK inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages is preferred. Parenteral administration, at high dosages, however, also can be used when safe and convenient for the patient.

Optimal dosages to be administered may be readily determined by those skilled in the art, and will vary with the particular hYAK inhibitor in use, the strength of the preparation, the mode of administration, and the advancement of the disease condition. Additional factors depending on the particular patient being treated will result in a need to adjust dosages, including patient age, weight, diet, and time of administration.

The method of this invention of inducing hYAK inhibitory activity in mammals, including humans, comprises administering to a subject in need of such activity an effective hYAK inhibiting amount of a pharmaceutically active compound of the present invention.

The invention also provides for the use of a compound of Formula (I) in the manufacture of a medicament for use as a hYAK inhibitor.

The invention also provides for the use of a compound of Formula (I) in the manufacture of a medicament for use in therapy.

The invention also provides for the use of a compound of Formula (I) in the manufacture of a medicament for use in treating diseases of the hematopoietic system, particularly anemias.

The invention also provides for a pharmaceutical composition for use as a hYAK inhibitor which comprises a compound of Formula (I) and a pharmaceutically acceptable carrier.

The invention also provides for a pharmaceutical composition for use in the treatment of diseases of the hematopoietic system, particularly anemias which comprises a compound of Formula (I) and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, such as other compounds known to treat diseases of the hematopoietic system, particularly anemias, or compounds known to have utility when used in combination with a hYAK inhibitor.

Without further elaboration, it is believed that one skilled in the art can, using the preceding description, utilize the present invention to its fullest extent. The following Examples are, therefore, to be construed as merely illustrative and not a limitation of the scope of the present invention in any way.

For ease of illustration, the regiochemistry around the double bonds in the chemical formulas in the Examples are drawn as fixed for ease of representation; however, a skilled in the art will readily appreciate that the compounds will naturally assume more thermodynamically stable structure around the C═N (the imine) double bond if it exits as exo form. Further compounds can also exit in endo form. As stated before, the invention contemplates both endo and exo forms as well as both regioisomers around the exo imine bond. Further it is intended that both E and Z isomers are encompassed around the C═C double bond.

### EXPERIMENTAL DETAILS

The compounds of Examples 1 to 6 are readily made according to Schemes I and II or by analogous methods.

### Example 1

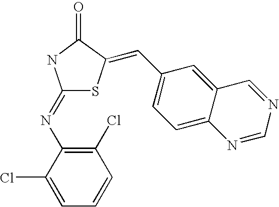

### (5Z)-2-[(2,6-Dichlorophenyl)amino]-5-(6-quinazolinylmethylidene)-1,3-thiazol-4(5H)-one

a) [(5-Chloro-2-nitrophenyl)methanediyl]diformamide. A slurry of 5-chloro-2-nitrobenzaldehyde (4 g, 21.6 mmol) in formamide (20 mL) was treated with HCl gas. An exothermic reaction resulted and the slurry dissolved rapidly. After 30 s. of bubbling HCl gas, a white precipitate formed and the HCl gas was removed. The r×n mixture was allowed to cool to RT over 1 h. Ethanol (50 mL) was added and the product collected via filtration to give a white powder. Recrystallization in boiling water followed by filtration yielded white needles (2.90 g, 52%) of the desired product. MS (ES+) m/e 258 [M+H]⁺.
b) 6-Chloroquinazoline. To a mixture of the compound obtained in example 1a (2.9 g, 11.3 mmol) and zinc dust (8.3 g, 184 mmol) in acetic acid (11 g, 184 mmol) was added crushed ice (30 g) over 10 min. The mixture was shaken for ½ h and stirred vigorously for a further 1½ h adding a total of 1.5 g of additional zinc dust portion wise. The reaction mixture was filtered and the filtrate basified with NaOH and a white cloudy solution resulted. The basic solution was extracted with ether (4×100 mL). The organic layer was dried over Na₂SO4, filtered and solvent removed under reduced pressure to give a white solid (500 mg, 27%) as the desired product. (ES+) m/e 165 [M+H]⁺.
c) 6-Ethenylquinazoline. The chloroquinazoline from example 1b (200 mg, 1.22 mmol), tributyl vinyl tin (392 μL, 1.34 mmol) and palladium tetrakis triphenylphosphine (141 mg, 0.122 mmol) in dioxane (2 mL) and DMF (3 drops) were stirred and heated in a microwave reactor at 150° C. for 20 min. Purification by flash-chromatography (silica gel, 20-50% ethyl acetate in hexanes) afforded the title compound (65 mg; 34%) as an off white solid. C₁₀H₈N₂ MS (ES+) m/e 157 [M+H]⁺
d) 6-Quinazolinecarbaldehyde. A mixture of the vinyl compound from example 1c (65 mg, 0.416 mmol), 2.5% osmium tetraoxide in t-butanol (85 mg, 0.008 mmol), sodium periodate (356 mg, 1.66 mmol) and 2,6-lutidene (97 μL, 0.833 mmol) in dioxane (3 mL) and water (1 mL) was stirred at RT for 30 min. The reaction mixture was diluted with water (10 mL) and extracted with DCM (2×30 mL). The organic layer was dried over MgSO₄, filtered and solvents removed under reduced pressure to yield the title compound (45 mg, 68%) which was used in the next step without further purification. C₉H₆N₂O MS (ES+) m/e 159 [M+H]⁺
e) (5Z)-2-[(2,6-Dichlorophenyl)amino]-5-(6-quinazolinylmethylidene)-1,3-thiazol-4(5H)-one. A solution of the compound from example 1d (45 mg, 0.284 mmol.), 2-[(2,6-dichlorophenyl)amino]-1,3-thiazol-4(5M-one (75 mg, 0.284 mmol.) and piperidine (28 μL, 0.284 mmol.) in ethanol (2.0 mL) was stirred and heated in a microwave reactor at 150° C. for 20 min. The mixture was purified by flash-chromatography (silica gel, 5-100% 10% methanol in chloroform) to afford the title compound (10.0 mg, 9%) as a pale-yellow powder. 1H NMR (400 MHz, DMSO-d₆) d ppm 13.10 (s, 1H) 9.72 (s, 1H) 9.32 (s, 1H) 8.28 (s, 1H) 8.09 (s, 2H) 7.92 (s, 1H) 7.57 (s, 2H) 7.24 (s, 1H). C₁₈H₁₀Cl₂N₄OS MS (ES+) m/e 401 [M+H]⁺

### Example 2

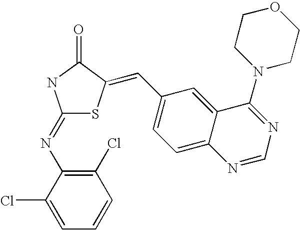

### (5Z)-2-[(2,6-Dichlorophenyl)amino]-5-{[4-(4-morpholinyl)-6-quinazolinyl]methylidene}-1,3-thiazol-4(5H)-one

a) 6-Ethenyl-4-(4-morpholinyl)quinazoline. A solution of 6-bromo-4-chloroquinazoline (147.7 mg, 0.608 mmol.) and morpholine (53 μL, 0.608 mmol.) in dioxane (2.0 mL) was stirred and heated in a microwave reactor at 150° C. for 20 min. An aliquot was analyzed by LCMS, MS (ES+) m/e 294 [M+H]⁺, and was 100% pure 6-bromo-4-(4-morpholinyl)quinazoline. Material used in next step without further workup. To the above mixture was added tributyl vinyl tin (195 μL, 0.608 mmol) and palladium tetrakis triphenylphosphine (70 mg, 0.0608 mmol) in DMF (2 mL). The reactants were stirred and heated in a microwave reactor at 150° C. for 20 min. Purification by flash-chromatography (silica gel, 10-100% 10% methanol in chloroform) afforded the title compound (125 mg; 85%) as an off white solid. C₁₄H₁₅N₃O MS (ES+) m/e 242 [M+H]⁺
b) 4-(4-Morpholinyl)-6-quinazolinecarbaldehyde. A mixture of the vinyl compound from example 2a (125 mg, 0.514 mmol), 2.5% osmium tetraoxide in t-butanol (105 mg, 0.010 mmol), sodium periodate (440 mg, 2.06 mmol) and 2,6-lutidene (120 μL, 1.03 mmol) in dioxane (6 mL) and water (1.5 mL) was stirred at RT for 30 min. The reaction mixture was diluted with water (10 mL) and extracted with DCM (2×30 mL). The organic layer was dried over MgSO₄, filtered and solvents removed under reduced pressure to yield the title compound (120 mg, 96%) as a clear oil which was used in the next step without further purification. C₁₃H₁₃N₃O₂ MS (ES+) m/e 244 [M+H]+
c) (5Z)-2-[(2,6-Dichlorophenyl)amino]-5-{[4-(4-morpholinyl)-6-quinazolinyl]methylidene}-1,3-thiazol-4(5H)-one. A solution of the aldehyde from example 2b (120 mg, 0.494 mmol.), 2-[(2,6-dichlorophenyl)amino]-1,3-thiazol-4(5H)-one (100 mg, 0.385 mmol.) and piperidine (40 μL, 0.385 mmol.) in ethanol (2.0 mL) was stirred and heated in a microwave reactor at 150° C. for 20 min. The mixture was purified by flash-chromatography (silica gel, 5-100% 10% methanol in chloroform) to afford the title compound (32.0 mg, 17%) as a yellow powder. ¹H NMR (400 MHz, DMSO-d₆) d ppm 13.04 (s, 1H) 8.64-8.69 (m, 1H) 8.02-8.08 (m, 1H) 7.90-7.97 (m, 2H) 7.83-7.89 (m, 1H) 7.54-7.62 (m, 2H) 7.20-7.29 (m, 1H) 3.64 (s, 8H). C₂₂H₁₇Cl₂N₅O₂S MS (ES+) m/e 486 [M+H]⁺

### Example 3

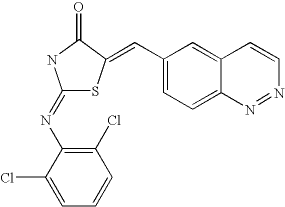

### (5Z)-5-(6-Cinnolinylmethylidene)-2-[(2,6-dichlorophenyl)amino]-1,3-thiazol-4(5H)-one

a) Methyl 4-amino-3-iodobenzoate. A solution of methyl 4-aminobenzoate (5.0 g; 0.033 mol.), benzyltrimethylammonium dichloroiodate (22.1 g; 0.056 mol.) and calcium carbonate (5.0 g; 0.050 mol.) in a mixture of dichloromethane (200 ml) and methanol (100 mL) were stirred and heated under reflux overnight. The solution was cooled, washed with saturated sodium bisulphate, dried and evaporated to afford the desired product (9.6 g; quant.) which was used directly without further purification. 1 H NMR (400 MHz, DMSO-d₆) δ ppm 3.75 (s, 3H) 6.09 (s, 2H) 6.75 (d, J=8.59 Hz, 1H) 7.66 (dd, J=8.59, 2.02 Hz, 1H) 8.11 (d, J=2.02 Hz, 1H).
b) Methyl 4-(3,3-diethyl-1-triazen-1-yl)-3-iodobenzoate. A suspension of the compound from example 3a) (9.6 g; 0.033 mol.) in acetonitrile (35.0 mL) was treated with ice (12.5 g) and concentrated hydrochloric acid (8.5 mL) and then cooled to −5° C. This suspension was then treated dropwise with a solution of sodium nitrite (5.3 g; 0.076 mol.) in a mixture of acetonitrile (10.0 mL) and water (30.0 mL). The solution was then stirred at −5° C. for 30 min. then added dropwise via cannula to a cooled (0° C.) solution of diethylamine (35.9 mL; 0.35 mol.) and potassium carbonate (29.0 g; 0.21 mol.) in acetonitrile (88 mL) and water (262 mL). The mixture was then stirred and allowed to reach room temperature overnight. The mixture was diluted with dichloromethane (500 mL), the layers separated and the organic layer washed with sat. aqu. sodium hydrogen carbonate, dried and evaporated. The residue was purified by chromatography [silica gel, hexanes/ethyl acetate (95:5) then (9:1)] to give the title compound (5.3 g; 42%) as an orange oil. 1H NMR (400 MHz, DMSO-d₆) δ ppm 1.26 (t, J=7.07 Hz, 3H) 1.31 (t, J=7.20 Hz, 3H) 3.79-3.89 (m, 7H) 7.38 (d, J=8.59 Hz, 1H) 7.90 (dd, J=8.46, 1.89 Hz, 1H) 8.35 (d, J=1.77 Hz, 1H).
c) Methyl 4-(3,3-diethyl-1-triazen-1-yl)-3-ethynylbenzoate. A solution of the compound from example 3b) (5.0 g; 0.014 mol.), trimethylsilylacetylene (2.8 ml; 0.021 mol.), bis(triphenylphosphine)dichloropalladium (0.59 g; 0.8 mmol.) and copper (I) iodide (0.32 g; 1.7 mmol.) in triethylamine (139 mL) was stirred and heated at 50° C. under an argon atmosphere overnight. The mixture was cooled and evaporated then the residue was filtered through a silica gel pad with the aid of dichloromethane/hexanes (1:1) (500 mL). After evaporation of the organics the crude residue was dissolved in tetrahydrofuran (100 mL) and methanol (20.0 mL) then treated with potassium carbonate (19.1 g; 0.139 mol.) and the mixture was stirred at room temperature overnight. The mixture was diluted with diethyl ether (500 mL) then washed with sat. aqueous ammonium chloride, dried and evaporated. The residue was then purified by chromatography [silica gel, hexanes/dichloromethane (3:1) then (2:1)] to afford the title compound (1.6 g; 44%) as a yellow oil. 1H NMR (400 MHz, DMSO-d₆) δ ppm 1.22 (t, J=7.07 Hz, 3H) 1.30 (t, J=7.20 Hz, 3H) 3.77-3.88 (m, 7H) 7.45 (d, J=8.59 Hz, 1H) 7.88 (dd, J=8.59, 2.02 Hz, 1H) 7.97 (d, J=2.02 Hz, 1H).
d) Methyl 6-cinnolinecarboxylate. A solution of the compound from example 3c) (1.6 g; 6.2 mmol.) in 1,3-dichlorobenzene (65.0 mL) was stirred and heated at 200° C. in a sealed vessel for 20 h. The mixture was colled, evaporated and the residue purified by chromatography [silica gel, hexanes/ethyl acetate (1:1)] to afford the title compound (0.36 g; 31%) as a brown powder. 1H NMR (400 MHz, DMSO-d₆) δ ppm 3.98 (s, 3H) 8.37 (dd, J=8.97, 1.89 Hz, 1H) 8.48 (d, J=5.05 Hz, 1H) 8.60 (d, J=8.84 Hz, 1H) 8.83 (d, J=1.77 Hz, 1H) 9.54 (d, J=5.81 Hz, 1H).
e) 6-Cinnolinecarbaldehyde. A solution of the compound from example 3d) (345 mg; 1.8 mmol.) in anhydrous tetrahydrofuran (20.0 mL) was cooled to 5° C. for the portionwise addition of solid lithium aluminumhydride (70.0 mg; 1.8 mmol.) and stirred at 5° C. for 1 h. The mixture was quenched by the addition of ethyl acetate (50.0 mL) then water (5.0 mL), filtered through a pad of Celite and evaporated. The residue was dissolved in ethyl acetate (50.0 mL) and treated with manganese dioxide (0.50 g) and stirred at room temperature for 2 h. The mixture was filtered through a pad of Celite and evaporated to afford the title compound (169 mg; 60%) as a brown powder which was used directly in the following step without further purification.
f) (5Z)-5-(6-Cinnolinylmethylidene)-2-[(2,6-dichlorophenyl)amino]-1,3-thiazol-4(5H)-one. A solution of the compound from example 3e) (158 mg; 1.0 mmol.), 2-[(2,6-dichlorophenyl)amino]-1,3-thiazol-4(5″-one (261 mg; 1.0 mmol.) and piperidine (0.11 mL; 1.1 mmol.) in ethanol (2.0 mL) was stirred and heated at 150° C. for 30 min. in a Biotage Initiator microwave synthesizer. The reaction mixture was then cooled and purified directly by chromatography [ODS silica, gradient elution with 10-100% acetonitrile/water (0.1% TFA)] to afford the title compound (53.0 mg; 7% over three steps from ethyl 6-cinnolinecarboxylate) as a brown powder. 1H NMR (400 MHz, DMSO-d₆) δ ppm 7.25 (t, J=8.08 Hz, 1H) 7.59 (d, J=8.08 Hz, 2H) 7.94 (s, 1H) 8.05 (dd, J=8.97, 1.64 Hz, 1H) 8.20 (d, J=1.64 Hz, 1H) 8.35 (d, J=5.81 Hz, 1H) 8.52 (d, J=8.84 Hz, 1H) 9.40 (d, J=6.06 Hz, 1H) 13.15 (s, 1H). C₁₈H₁₀N₄OSCl₂.0.5H₂O requires: % C, 52.7; % H, 2.7; % N, 13.6. found: % C, 53.2; % H, 2.7; % N, 13.1.

### Example 4

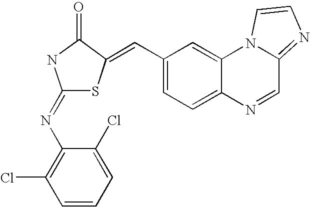

### (5Z)-2-[(2,6-Dichlorophenyl)amino]-5-(imidazo[1,2-a]quinoxalin-8-ylmethylidene)-1,3-thiazol-4(5H)-one

a) 2-[(2,6-Dichlorophenyl)amino]-1,3-thiazol-4(5H)-one. A suspension of N-(2,6-dichlorophenyl)thiourea (103.7 g; 0.469 mol.) and chloroacetic acid (48.8 g; 0.516 mol.) in glacial acetic acid (600 mL) was stirred and heated under reflux for 2 h. The stirred mixture was allowed to cool to 40° C. then treated dropwise with water (1 L) during which a pale-yellow precipitate formed. The suspension was then filtered and the precipitate washed with water (1 L) to afford the title compound (94.0 g; 79%) as a yellow solid. 1H NMR (400 MHz, DMSO-d₆) δ ppm 12.2 (s, 1H) 7.49 (d, J=8.08 Hz, 2H) 7.14 (t, J=8.08 Hz, 1H) 4.10 (s, 2H).
b) 7-Bromo-2-chloroquinoxaline. A suspension of 7-bromo-2(1H)-quinoxalinone (prepared by the method of Linda, P; Marino, G. Ric. Sci. Rend., Ser. A. 1963, 3, 225-228) (24.0 g; 107 mmol) in phosphorus oxychloride (40.0 mL) was stirred and heated under reflux for 2 h. The stirred mixture was allowed to cool then carefully poured into ice-water. The suspension was then filtered and the precipitate washed with water to afford the title compound (25.0 g; 96%) as a beige solid. ¹H NMR (400 MHz, DMSO-d₆) δ ppm 9.05 (s, 1H) 8.34 (d, J=2.0 Hz, 1H) 8.11 (d, J=9.0 Hz, 1H) 8.05 (dd, J=8.9, 2.0 Hz, 1H).
c) 8-Bromoimidazo[1,2-a]quinoxaline. A solution of the compound from Example 1b) (0.243 g; 1.00 mmol) and aminoacetaldehyde dimethyl acetal (0.119 mL; 1.10 mmol) in methanol (2.0 mL) was irradiated at 150° C. for 20 min. in a Biotage Initiator microwave synthesizer. Upon cooling, 6 M aq. HCl (1.0 mL) was added and the reaction mixture was irradiated at 120° C. for 20 min. in a Biotage Initiator microwave synthesizer. Upon cooling the reaction mixture was poured into a solution of saturated aq. NaHCO₃, diluted with water, and extracted thrice with ethyl acetate. The combined organic layers were dried over MgSO₄, filtered, and concentrated in vacuo to afford the title compound (0.200 g; 81%) as an orange solid. ¹H NMR (400 MHz, CHLOROFORM-d) □ ppm 9.12 (s, 1H) 8.07-8.10 (m, 2H) 8.00 (d, J=8.8 Hz, 1H) 7.84 (d, J=1.3 Hz, 1H) 7.71 (dd, J=8.8, 2.0 Hz, 1H).
d) 8-Ethenylimidazo[1,2-a]quinoxaline. A solution of the compound from Example 1c) (0.200 g; 0.806 mmol), tributyl(vinyl)tin (0.259 mL; 0.887 mmol), and tetrakis(triphenylphosphine)palladium (0.009 g; 0.008 mmol) in dioxane (2.0 mL) and N,N-dimethylformamide (3 drops) was irradiated at 150° C. for 20 min. in a Biotage Initiator microwave synthesizer. Upon cooling, the solution was concentrated onto silica gel and purified via flash column chromatography (silica gel, 50-80% ethyl acetate in hexanes) to afford the title compound (0.138 g; 88%) as a tan solid. ¹H NMR (400 MHz, CHLOROFORM-d) □ ppm 9.05 (s, 1H) 8.11 (s, 1H) 8.03 (d, J=8.6 Hz, 1H) 7.81-7.82 (m, 1H) 7.80 (d, J=1.3 Hz, 1H) 7.65 (dd, J=8.6, 1.8 Hz, 1H) 6.87 (dd, J=17.6, 11.0 Hz, 1H) 5.95 (d, J=17.7 Hz, 1H) 5.47 (d, J=10.9 Hz, 1H).
e) Imidazo[1,2-a]quinoxaline-8-carbaldehyde. To a suspension of the compound from Example 1d) (0.100 g; 0.512 mmol) in dioxane (3.0 mL) and water (1.0 mL) was added osmium tetroxide (2.5% soln. in t-BuOH; 0.104 g; 0.010 mmol), sodium periodate (0.438 g; 2.05 mmol), and 2,6-lutidine (0.119 mL; 1.02 mmol). After stirring 30 min. at ambient temperature, water was added and the reaction mixture was extracted twice with CH₂Cl₂. The combined organic layers were dried over MgSO₄, filtered, and concentrated in vacuo to afford the title compound (0.091 g; 90%) as a light yellow solid. ¹H NMR (400 MHz, CHLOROFORM-D) □ ppm 10.2 (s, 1H) 9.25 (s, 1H) 8.47 (d, J=1.3 Hz, 1H) 8.30 (d, J=8.3 Hz, 1H) 8.27 (s, 1H) 8.10 (dd, J=8.2, 1.4 Hz, 1H) 7.92 (s, 1H).
f) (5Z)-2-[(2,6-Dichlorophenyl)amino]-5-(imidazo[1,2-a]quinoxalin-8-ylmethylidene)-1,3-thiazol-4(5″-one. A suspension of the compound from Example 1a) (0.093 g; 0.355 mmol), the compound from Example 1e) (0.070 g; 0.355 mmol), and piperidine (0.035 mL; 0.355 mmol) in ethanol (2.0 mL) was stirred and irradiated at 150° C. for 30 min. in a Biotage Initiator microwave synthesizer. Upon cooling, the reaction mixture was diluted with water and acidified with 1 M aq. HCl. The resulting suspension was filtered, washed with water, and dried in vacuo to afford the title compound (0.105 g, 67%) as a yellow solid. ¹H NMR (400 MHz, DMSO-d₆) □ ppm 13.1 (s, 1H) 9.15 (s, 1H) 8.75 (s, 2H) 8.14 (d, J=8.6 Hz, 1H) 7.90 (d, J=5.1 Hz, 2H) 7.62 (dd, J=8.6, 1.3 Hz, 1H) 7.58 (d, J=8.1 Hz, 2H) 7.24 (t, J=8.2 Hz, 1H).

### Example 5

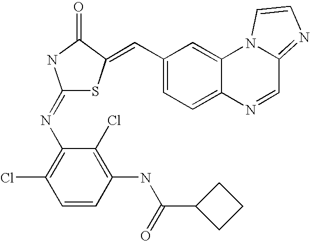

### N-(4-Chloro-3-{[(5Z)-5-(imidazo[1,2-a]quinoxalin-8-ylmethylidene)-4-oxo-4,5-dihydro-1,3-thiazol-2-yl]amino}phenyl)cyclobutanecarboxamide

a) N-(3-Amino-4-chlorophenyl)cyclobutanecarboxamide. Cyclobutylcarbonyl chloride (3.64 mL, 31.9 mmol) was added dropwise to an ice-cooled, stirred solution of 4-chloro-3-nitroaniline (5.00 g, 29.0 mmol) and pyridine (3.5 mL, 43.2 mmol) in dichloromethane (30 mL) under argon. The mixture was allowed to warm to room temperature and stirred 1 h, then the solvent removed under reduced pressure. 0.5 M aqueous potassium carbonate (10 mL) and methanol (30 mL) was added and the mixture stirred 0.5 h, then diluted with water (200 mL) and extracted with ethyl acetate. The extracts were washed (1 M aq HCl, water, brine), dried (MgSO₄) and evaporated to dryness under reduced pressure. A solution of the crude amide in methanol (150 mL) was stirred with Raney® nickel (˜0.5 g) under 1 atm of hydrogen for 18 h. After removal of the hydrogen, the mixture was filtered through a PTFE micropore filter, then evaporated under reduced pressure to give the title compound (6.52 g, 100%) as an oil. 1H NMR (400 MHz, DMSO-d₆) δ1.80 (m, 1H), 1.92 (m, 1H), 2.03-2.11 (m, 2H), 2.14-2.24 (m, 2H), 3.18 (m, 1H), 5.32 (s, 2H), 6.73 (dd, J=8.6, 2.5 Hz, 1H), 7.05 (d, J=8.6 Hz, 1H), 7.20 (d, J=2.5 Hz, 1H), 9.56 (s, 1H).
b) N-{4-Chloro-3-[(4-oxo-4,5-dihydro-1,3-thiazol-2-yl)amino]phenyl}cyclobutane-carboxamide. A mixture of the compound from example 47(a) (6.55 g, 29.0 mmol), 2-(methylthio)-1,3-thiazol-4(5H)-one (A. I. Khodair, J. Heterocyclic Chem., 2002, 39, 1153; 5.30 g, 36.0 mmol) and ethanol (36 mL) was heated under reflux for 18 h, then cooled. The solid was filtered, washed with ethanol and dried to give the title compound (8.33 g, 89%) as a brown solid. 1H NMR (400 MHz, DMSO-d₆) δ1.82 (m, 1H), 1.95 (m, 1H), 2.10 (m, 2H), 2.21 (m, 2H), 3.21 (m, 1H), 4.03 (s, 2H), 7.30 (d, J=8.8 Hz, 1H), 7.38 (d, J=8.8 Hz, 1H), 7.45 (s, 1H), 9.85 (s, 1H), 11.99 (br s, 1H).
c) Following the procedure of Example 1f), except substituting the compound from example 5(b) for the compound from Example 1a), the title compound was obtained as a brown solid. ¹H NMR (400 MHz, DMSO-d₆) □ ppm 12.8 (s, 1H) 9.96 (s, 1H) 9.15 (s, 1H) 8.76 (s, 1H) 8.72 (d, J=1.0 Hz, 1H) 8.14 (d, J=8.6 Hz, 1H) 7.88 (d, J=17.2 Hz, 2H) 7.66 (dd, J=8.3, 1.3 Hz, 1H) 7.51 (d, J=1.0 Hz, 1H) 7.46 (d, J=8.8 Hz, 1H) 7.41 (dd, J=8.4, 2.0 Hz, 1H) 3.19-3.28 (m, 1H) 2.15-2.26 (m, 2H) 2.03-2.14 (m, 2H) 1.85-1.99 (m, 1H) 1.72-1.84 (m, 1H).

### Example 6

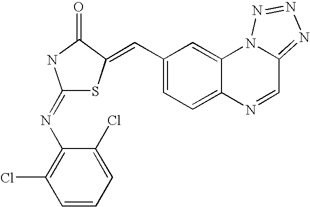

### (5Z)-2-[(2,6-Dichlorophenyl)amino]-5-(tetrazolo[1,5-a]quinoxalin-8-ylmethylidene)-1,3-thiazol-4(5H)-one

a) 8-Bromotetrazolo[1,5-a]quinoxaline. A solution of the compound from Example 1b) (0.430 g; 1.77 mmol) and sodium azide (0.138 g; 2.12 mmol) in N,N-dimethylformamide (5.0 mL) was heated to 120° C. for 2 h. Upon cooling to ambient temperature, water was added and the resulting precipitate was collected by filtration, washed with water, and dried in vacuo to afford the title compound (0.403 g; 91%) as a light orange solid. ¹H NMR (400 MHz, CHLOROFORM-d) □ ppm 9.58 (s, 1H) 8.84 (d, J=2.0 Hz, 1H) 8.20 (d, J=8.8 Hz, 1H) 7.99 (dd, J=8.7, 2.1 Hz, 1H).
b) 8-Ethenyltetrazolo[1,5-a]quinoxaline. Following the procedure of Example 1d), except substituting the compound from Example 6a) for the compound from Example 1c), the title compound was obtained as an off-white solid. ¹H NMR (400 MHz, CHLOROFORM-d) □ ppm 9.51 (s, 1H) 8.60 (d, J=1.8 Hz, 1H) 8.25 (d, J=8.6 Hz, 1H) 7.92 (dd, J=8.6, 1.8 Hz, 1H) 6.97 (dd, J=17.6, 11.0 Hz, 1H) 6.12 (d, J=17.4 Hz, 1H) 5.63 (d, J=10.9 Hz, 1H).
c) Tetrazolo[1,5-a]quinoxaline-8-carbaldehyde. Following the procedure of Example 1e), except substituting the compound from Example 6b) for the compound from Example 1d), the title compound was obtained as a light yellow solid. ¹H NMR (400 MHz, CHLOROFORM-D) □ ppm 10.3 (s, 1H) 9.70 (s, 1H) 9.14 (d, J=1.5 Hz, 1H) 8.51 (d, J=8.6 Hz, 1H) 8.38 (dd, J=8.5, 1.6 Hz, 1H).
d) (5Z)-2-[(2,6-Dichlorophenyl)amino]-5-(tetrazolo[1,5-a]quinoxalin-8-ylmethylidene)-1,3-thiazol-4(5″-one. Following the procedure of Example 1f), except substituting the compound from Example 6c) for the compound from Example 1e), the title compound was obtained following purification via flash column chromatography (silica gel, 10-100% ethyl acetate in hexanes) as a yellow solid. ¹H NMR (400 MHz, CHLOROFORM-d) □ ppm 9.59 (s, 1H) 8.64 (d, J=1.0 Hz, 1H) 8.35 (d, J=8.6 Hz, 1H) 7.96 (s, 1H, 7.89 (dd, J=8.3, 1.3 Hz, 1H) 7.42 (d, J=8.3 Hz, 2H) 7.14 (t, J=8.2 Hz, 1H).

### Example 7

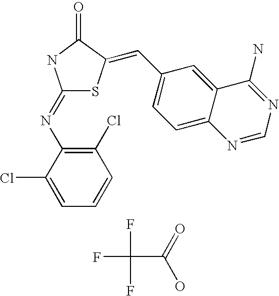

### (5Z)-5-[(4-Amino-6-quinazolinyl)methylidene]-2-[(2,6-dichlorophenyl)amino]-1,3-thiazol-4(5H)-one trifluoroacetate

a) 6-Bromo-4-quinazolinamine. Ammonia gas was bubbled through a solution of 6-bromoisotoic anhydride (2 g, 8.26 mmol) in DMF (20 mL) at r.t. for 15 min. The solution was then degassed by bubbling nitrogen into the solution for 5 min also removing (NH₄)CO₃ which was formed. The resulting solution was cooled to 0° C. and POCl₃ (2 mL) was added dropwise. Upon complete addition the reaction was warmed to 50° C. for 30 minutes and then cooled to r.t. To the cooled solution were added water (6 mL) and 35% ammonium hydroxide (9 mL). The resulting solution was heated at 100° C. for 1 h and then allowed to cool to r.t. where a white precipitate formed. The solid was collected by filtration to give the product as a white solid (1.51 g, 82%). C₈H₆BrN₃ MS (ES+) m/e 224 [M+H]⁺
b) 6-Ethenyl-4-quinazolinamine. To 6-bromo-4-quinazolinamine (1 g, 4.48 mmol) in DMF (4 mL) was added tributyl vinyl tin (1.44 mL, 4.93 mmol) and palladium tetrakis triphenylphosphine (260 mg, 0.224 mmol). The reactants were stirred and heated in a microwave reactor at 150° C. for 25 min. Purification by flash-chromatography (silica gel, 10-100% 10% methanol in chloroform) afforded the title compound (392 mg; 51%) as a white solid. C₁₀H₉N₃ MS (ES+) m/e 172 [M+H]⁺
c) 4-Amino-6-quinazolinecarbaldehyde. A mixture of the vinyl compound (6-ethenyl-4-quinazolinamine) from example 7b (292 mg, 1.69 mmol), 2.5% osmium tetraoxide in t-butanol (345 mg, 0.034 mmol), sodium periodate (1.45 g, 6.79 mmol) and 2,6-lutidene (395 μL, 3.39 mmol) in dioxane (9 mL) and water (3 mL) was stirred at RT for 30 min. The reaction mixture was diluted with water (10 mL) and extracted with DCM (2×30 mL). The organic layer was dried over MgSO₄, filtered and solvents removed under reduced pressure to yield the title compound (250 mg, 85%) as a clear oil which was used in the next step without further purification. C₉H₇N₃O MS (ES+) m/e 174 [M+H]⁺
d) (5Z)-5-[(4-Amino-6-quinazolinyl)methylidene]-2-[(2,6-dichlorophenyl)amino]-1,3-thiazol-4(5H)-one. A solution of crude 4-amino-6-quinazolinecarbaldehyde from Example 7c (200 mg, 1.17 mmol.), 2-[(2,6-dichlorophenyl)amino]-1,3-thiazol-4(5H)-one (100 mg, 0.385 mmol.) and piperidine (40 μL, 0.385 mmol.) in ethanol (3.0 mL) was stirred and heated in a microwave reactor at 150° C. for 20 min. The mixture was purified by HPLC chromatography (ODS silica, gradient 10-100% acetonitrile/water (0.1% TFA)) to afford the title compound (11.0 mg, 7%) as a yellow powder. 1H NMR (400 MHz, DMSO-d₆) □ ppm 13.12 (s, 1H) 9.44 (s, 2H) 8.74 (s, 1H) 8.50 (d, J=1.26 Hz, 1H) 7.98 (d, J=8.59 Hz, 1H) 7.82 (d, J=8.59 Hz, 1H) 7.73 (s, 1H) 7.58 (d, J=8.08 Hz, 2H) 7.24 (t, J=8.08 Hz, 1H).

### Example 8

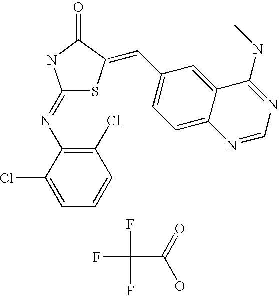

### (5Z)-2-[(2,6-Dichlorophenyl)amino]-5-{[4-(methylamino)-6-quinazolinyl]methylidene}-1,3-thiazol-4(5H)-one trifluoroacetate

The title compound was prepared following the procedure of example 7a-d except substituting methylamine for ammonium hydroxide. The final compound was purified by HPLC chromatography (ODS silica, gradient 10-100% acetonitrile/water (0.1% TFA)) to afford a yellow powder (12.0 mg, 7%). 1H NMR (400 MHz, DMSO-d₆) □ ppm 13.13 (s, 1H) 10.07 (s, 1H) 8.87 (s, 1H) 8.52 (s, 1H) 7.98 (d, J=8.34 Hz, 1H) 7.85 (d, J=8.59 Hz, 1H) 7.74 (s, 1H) 7.58 (d, J=8.08 Hz, 2H) 7.24 (t, J=8.21 Hz, 1H) 3.18 (d, J=4.04 Hz, 3H). C₁₉H₁₃Cl₂N₅OS MS (ES+) m/e 430 [M+H]⁺

### Example 9

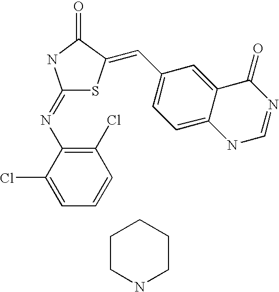

### 6-{(Z)-[2-[(2,6-Dichlorophenyl)amino]-4-oxo-1,3-thiazol-5(4H)-ylidene]methyl}-4(1H)-quinazolinone, piperidine salt

a) 4-Oxo-1,4-dihydro-6-quinazolinecarbaldehyde. A mixture of the vinyl compound (6-ethenyl-4(1H-quinazolinone) obtained as a minor product from example 3a (400 mg, 2.32 mmol), 2.5% osmium tetraoxide in t-butanol (472 mg, 0.046 mmol), sodium periodate (994 mg, 4.65 mmol) and 2,6-lutidene (458 μL, 4.65 mmol) in dioxane (14 mL) and water (4 mL) was stirred at RT for 30 min. The reaction mixture was diluted with water (10 mL) and extracted with DCM (2×30 mL). The organic layer was dried over MgSO₄, filtered and solvents removed under reduced pressure to yield the title compound (90 mg, 22%) as a clear oil which was used in the next step without further purification. C₉H₆N₂O₂ MS (ES+) m/e 174 [M+H]⁺
b) 6-{(Z)-[2-[(2,6-Dichlorophenyl)amino]-4-oxo-1,3-thiazol-5(4H)-ylidene]methyl}-4(1H)-quinazolinone. A solution of crude 44-oxo-1,4-dihydro-6-quinazolinecarbaldehyde from Example 9a (90 mg, 0.517 mmol.), 2-[(2,6-dichlorophenyl)amino]-1,3-thiazol-4(5H)-one (134 mg, 0.517 mmol.) and piperidine (50 μL, 0.517 mmol.) in ethanol (2.0 mL) was stirred and heated in a microwave reactor at 150° C. for 20 min. The mixture was poured into water (20 mL) and the precipitate formed was collected and dried via filtration to afford the title compound (71.0 mg, 33%) as a piperidine salt which was light brown. 1H NMR (400 MHz, DMSO-d₆) □ ppm 9.67 (s, 1H) 8.09 (s, 2 H) 7.87 (s, 1H) 7.69 (s, 1H) 7.36 (s, 3H) 7.02 (s, 1H) 2.96-3.04 (m, 4H) 1.60-1.67 (m, J=5.94, 5.62, 5.46, 5.46 Hz, 4H) 1.54 (dq, J=5.43, 5.18 Hz, 2H).

### Example 10

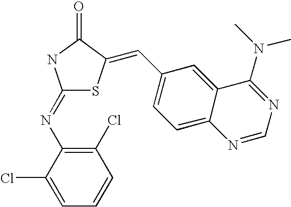

### (5Z)-2-[(2,6-Dichlorophenyl)amino]-5-{[4-(dimethylamino)-6-quinazolinyl]methylidene}-1,3-thiazol-4(5H)-one

The title compound was prepared following the procedure of example 3a-c except substituting dimethylamine for morpholine. The final compound was purified by flash-chromatography (silica gel, 10-100% 10% methanol in chloroform) to afford the title compound (14.2 mg; 4%) as a light brown solid ¹H NMR (400 MHz, DMSO-d₆) □ ppm 13.00 (s, 1H) 8.50 (s, 1H) 8.39 (s, 1H) 7.97 (s, 1H) 7.77 (s, 2H) 7.58 (d, J=8.08 Hz, 2H) 7.25 (t, J=8.08 Hz, 1H) 3.35 (s, 6H).

### Example 11

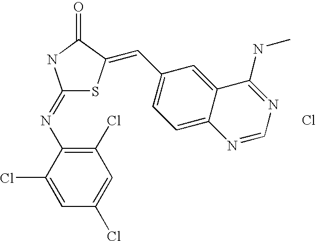

### (5Z)-5-{[4-(Methylamino)-6-quinazolinyl]methylidene}-2-[(2,4,6-trichlorophenyl)amino]-1,3-thiazol-4(5H)-one hydrochloride

The title compound was prepared following the procedure of example 7a-d except substituting 2-[(2,4,6-trichlorophenyl)amino]-1,3-thiazol-4(5H)-one for 2-[(2,6-dichlorophenyl)amino]-1,3-thiazol-4(5″-one. The final compound was purified by HPLC chromatography (ODS silica, gradient 10-100% acetonitrile/water (0.1% TFA)) to afford a yellow powder (60.2.0 mg, 15%). ¹H NMR (400 MHz, DMSO-d₆) □ ppm 13.17 (s, 1H) 10.13 (s, 1H) 8.90 (s, 1H) 8.53 (s, 1H) 8.02 (d, J=11.37 Hz, 1H) 7.85 (d, J=8.84 Hz, 1H) 7.82 (s, 2H) 7.74 (s, 1H) 3.19 (d, J=4.55 Hz, 3H).

### Example 12

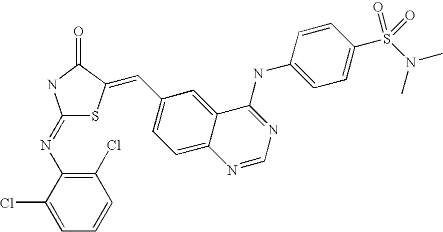

### 4-[(6-{(Z)-[2-[(2,6-Dichlorophenyl)amino]-4-oxo-1,3-thiazol-5(4H)-ylidene]methyl}-4-quinazolinyl)amino]-N,N-dimethylbenzenesulfonamide

The title compound was prepared following the procedure of example 7a-d except substituting 4-amino-N,N-dimethylbenzenesulfonamide for ammonium hydroxide. The final compound was purified by HPLC chromatography (ODS silica, gradient 10-100% acetonitrile/water (0.1% TFA)) to afford a yellow powder (6.0 mg, 3%). 1H NMR (400 MHz, DMSO-d₆) □ ppm 13.06 (s, 1H) 10.17 (s, 1H) 8.87 (s, 1H) 8.76 (s, 1H) 8.21 (d, J=8.84 Hz, 2H) 7.84-7.94 (m, 3H) 7.81 (d, J=8.84 Hz, 2H) 7.58 (d, J=8.08 Hz, 2H) 7.24 (s, 1H) 2.64 (s, 6H).

### Example 13

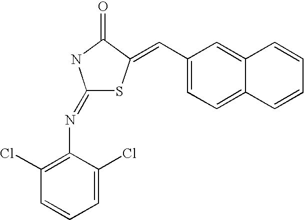

### (5Z)-2-[(2,6-Dichlorophenyl)amino]-5-(2-naphthalenylmethylidene)-1,3-thiazol-4(5H)-one

A mixture of 2-naphthalenecarbaldehyde (100 mg, 0.64 mmol), 2-[(2,6-dichlorophenyl)amino]-1,3-thiazol-4(5″-one (167 mg, 0.64 mmol) and piperidine (63 μL, 0.064 mmol) in ethanol (2 mL) was heated to 150° C. for half hour in a Biotage Initiator Synthesizer. The product was cooled, concentrated and purified via flash chromatography (0-10% methanol in methylene chloride) to afford the title compound as a yellow solid (90 mg, 35%). 1H NMR (400 MHz, DMSO-d₆) m ppm 12.98 (br. s., 1H) 8.11 (s, 1H) 7.97-8.03 (m, 2H) 7.93 (d, J=7.83 Hz, 1H) 7.88 (s, 1H) 7.52-7.63 (m, 5H) 7.24 (t, J=8.08 Hz, 1H)

### Example 14

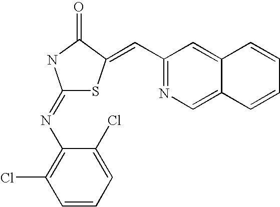

### (5Z)-2-[(2,6-Dichlorophenyl)amino]-5-(3-isoquinolinylmethylidene)-1,3-thiazol-4(5H)-one

Following the procedure of Example 1f), except substituting 3-isoquinolinecarbaldehyde (prepared by the method of Guanti, G.; Riva, R. Tet. Asymm. 2001, 12, 1185-1200) for the compound from Example 1e), the title compound was obtained as a beige solid. ¹H NMR (400 MHz, DMSO-d₆) □ ppm 12.7 (s, 1H) 9.37 (s, 1H) 8.29 (s, 1H) 8.08 (d, J=8.1 Hz, 1H) 8.00 (d, J=8.1 Hz, 1H) 7.86 (s, 1H) 7.84 (t, J=7.2 Hz, 1H) 7.75 (t, J=7.2 Hz, 1H) 7.57 (d, J=8.1 Hz, 2H) 7.23 (t, J=8.1 Hz, 1H).

### Example 15

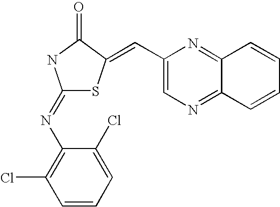

(5Z)-2-[(2,6-Dichlorophenyl)amino]-5-(2-quinoxalinylmethylidene)-1,3-thiazol-4(5H)-one
a) 2-Quinoxalinecarbaldehyde. To a solution of ethyl 2-quinoxalinecarboxylate (0.303 g; 1.50 mmol) in CH₂Cl₂ (10.0 mL) at −78° C. was added diisobutylaluminum hydride (1 M soln. in CH₂Cl₂; 1.50 mL; 1.50 mmol). After stirring 1 h at −78° C., the reaction was quenched by the addition of methanol. A solution of saturated aq. Na/K tartrate was added and the reaction mixture was extracted thrice with CH₂Cl₂. The combined organic layers were dried over MgSO₄, filtered, and concentrated in vacuo. Purification via flash column chromatography (silica gel, 10-20% ethyl acetate in hexanes) afforded the title compound (0.123 g; 52%) as an off-white solid.
b) (5Z)-2-[(2,6-Dichlorophenyl)amino]-5-(2-quinoxalinylmethylidene)-1,3-thiazol-4(5H)-one. Following the procedure of Example 1f), except substituting the compound from Example 15a) for the compound from Example 1e), the title compound was obtained as a dull yellow solid. ¹H NMR (400 MHz, DMSO-d₆) □ ppm 13.0 (s, 1H) 9.29 (s, 1H) 8.09 (d, J=7.1 Hz, 1H) 8.05 (s, 1H) 7.77-7.88 (m, 2H) 7.61 (d, J=8.1 Hz, 2H) 7.49 (d, J=8.1 Hz, 1H) 7.27 (t, J=8.2 Hz, 1H).

### Example 16

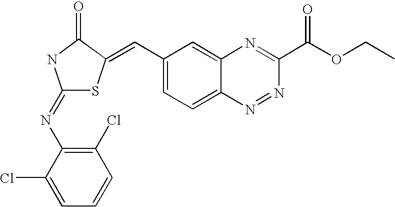

### Ethyl 6-{(Z)-[2-[(2,6-dichlorophenyl)amino]-4-oxo-1,3-thiazol-5(4H)-ylidene]methyl}-1,2,4-benzotriazine-3-carboxylate

a) Methyl [(4-bromo-2-nitrophenyl)hydrazono](nitro)ethanoate. A slurry of 4-bromo-2-nitroaniline (30.4 g; 0.14 mol.) in 2M aq. nitric acid (350 mL) was cooled to 5° C. then treated dropwise with a solution of sodium nitrite (9.7 g; 0.14 mol.) in water (75.0 mL). The solution was stirred at 5° C. for 15 min., quickly filtered then added dropwise to a suspension of methyl nitroacetate (6.3 g; 0.053 mol.) and sodium acetate (90.0 g) in water 200 mL) and ethanol (200 mL). The mixture was stirred at room temperature for 10 min. then filtered and the solid washed with water to afford the title compound (16.3 g; 89%) as a yellow solid. 1H NMR (400 MHz, DMSO-d₆) □ ppm 3.98 (s, 3H) 7.83 (d, J=9.09 Hz, 1H) 8.06 (dd, J=9.09, 2.02 Hz, 1H) 8.41 (d, J=2.02 Hz, 1H) 13.24 (s, 1H).
b) Methyl 6-bromo-1,2,4-benzotriazine-3-carboxylate. A solution of the compound from Example 22a) (6.4 g; 18 mmol.) in N,N-dimethylformamide (50.0 mL) was hydrogenated over 5% platinum-on-carbon (0.50 g) at room temperature and atmospheric pressure for 6 h. The mixture was filtered through a Celite pad and left overnight. The filtrate was evaporated and the residue purified by chromatography (silica gel, 0-5% methanol in chloroform) to afford a brown solid (3.2 g). A portion of this brown solid (1.3 g) was dissolved in N,N-dimethylformamide (10.0 mL), treated with 10% palladium-on-charcoal (0.30 g) and stirred at room temperature for 6 h. The mixture was filtered through a Celite pad and the filtrate was evaporated and the residue purified by chromatography (silica gel, 0-5% methanol in chloroform) to afford the title compound (0.262 g; 13% as an orange solid. 1H NMR (400 MHz, DMSO-d₆) □ ppm 4.07 (s, 3H) 8.33 (dd, J=9.09, 2.02 Hz, 1H) 8.62 (s, 1H) 8.65 (dd, J=2.02, 1.52 Hz, 1H).
c) Methyl 6-ethenyl-1,2,4-benzotriazine-3-carboxylate. A solution of the compound from Example 22b, (248 mg; 0.93 mmol.), tributylvinylstannane (370 mg; 1.2 mmol.) and tetrakis(triphenylphosphino)palladium (100 mg) in 1,4-dioxane (2.0 mL) and N,N-dimethylformamide (0.50 mL) was stirred and heated in a microwave reactor at 150° C. for 30 min. The mixture was cooled and purified by chromatography (silica gel, gradient 0-100% ethyl acetate in hexanes) to afford the title compound (85.0 mg; 43%) as a yellow solid. ¹H NMR (400 MHz, DMSO-d₆) □ ppm 4.06 (s, 3H) 5.76 (d, J=11.12 Hz, 1H) 6.42 (d, J=17.68 Hz, 1H) 7.12 (dd, J=17.68, 10.86 Hz, 1H) 8.30 (d, J=1.52 Hz, 1H) 8.44 (dd, J=8.97, 1.89 Hz, 1H) 8.64 (d, J=8.84 Hz, 1H).
d) Methyl 6-formyl-1,2,4-benzotriazine-3-carboxylate. A solution of the compound from Example 22c) (76.9 mg; 0.36 mmol.), sodium periodate (153 mg; 0.72 mmol.), osmium tetroxide (2 mg) and 2,6-lutidine (0.042 mL; 0.72 mmol.) in 1,4-dioxane (2.0 mL) and water (0.50 mL) was stirred at room temperature for 3 h. The mixture was diluted with water (25.0 mL) and extracted with ethyl acetate (2×50.0 mL), washed with sat. aq. NaCl (50.0 mL), dried and evaporated to give a brown solid. This solid was dissolved in methanol (4.0 mL) and water (1.0 mL), treated with sodium periodate 9300 mg) and stirred at room temperature overnight. The mixture was diluted with water (25.0 mL) and extracted with ethyl acetate (2×50.0 mL), washed with sat. aq. NaCl (50.0 mL), dried and evaporated to give the title compound (70.0 mg; 90%) as a yellow solid. 1H NMR (400 MHz, DMSO-d₆) □ ppm 4.09 (s, 3H) 8.48 (dd, J=8.72, 1.64 Hz, 1H) 8.86 (d, J=8.84 Hz, 1H) 8.94 (d, J=1.01 Hz, 1H) 10.37 (s, 1H).
e) Ethyl 6-{(Z)-[2-[(2,6-dichlorophenyl)amino]-4-oxo-1,3-thiazol-5(4H)-ylidene]methyl}-1,2,4-benzotriazine-3-carboxylate. A suspension of 2-[(2,6-dichlorophenyl)amino]-1,3-thiazol-4(5H)-one (85 mg; 0.33 mmol.), the crude compound from Example 22d) (70 mg; 0.32 mmol.) and piperidine (0.40 mL) in ethanol (2.0 mL) was stirred and heated in a microwave reactor at 150° C. for 20 min. The mixture was cooled and poured into 1 M aq. Hydrochloric acid. The mixture was extracted with ethyl acetate (2×), dried and evaporated and the residue purified by chromatography (ODS silica gel, 10-90% acetonitrile in water (0.01% TFA)) to afford the title compound (15.0 mg; 10%) as a yellow solid. ¹H NMR (400 MHz, DMSO-d₆) □ ppm 1.42 (t, J=7.07 Hz, 3H) 4.53 (q, J=7.24 Hz, 2H) 7.26 (t, J=8.08 Hz, 1H) 7.60 (d, J=8.08 Hz, 2H) 8.06 (s, 1H) 8.24 (dd, J=8.84, 1.77 Hz, 1H) 8.34 (d, J=1.26 Hz, 1H) 8.73 (d, J=8.84 Hz, 1H) 13.28 (s, 1H).

### Example 17

### Capsule Composition

An oral dosage form for administering the present invention is produced by filing a standard two piece hard gelatin capsule with the ingredients in the proportions shown in Table I, below.

TABLE I

| INGREDIENTS | AMOUNTS | |
|---|---|---|
| (5Z)-5-(6-Cinnolinylmethylidene)-2-[(2,6- | 25 | mg |
| dichlorophenyl)amino]-1,3-thiazol-4(5H)-one | | |
| Lactose | 55 | mg |
| Talc | 16 | mg |
| Magnesium Stearate | 4 | mg |

### Example 18

### Injectable Parenteral Composition

An injectable form for administering the present invention is produced by stirring 1.5% by weight of (5Z)-2-[(2,6-Dichlorophenylyamino]-5-(6-quinazolinylmethylidene)-1,3-thiazol-4(5H)-one in 10% by volume propylene glycol in water.

### Example 25

### Tablet Composition

The sucrose, calcium sulfate dihydrate and an hYAK inhibitor as shown in Table II below, are mixed and granulated in the proportions shown with a 10% gelatin solution. The wet granules are screened, dried, mixed with the starch, talc and stearic acid, screened and compressed into a tablet.

TABLE II

| INGREDIENTS | AMOUNTS | |
|---|---|---|
| (5Z)-2-[(2,6-Dichlorophenyl)amino]-5-{[4- | 20 | mg |
| (4-morpholinyl)-6-quinazolinyl]methylidene}- | | |
| 1,3-thiazol-4(5H)-one | | |
| calcium sulfate dihydrate | 30 | mg |
| sucrose | 4 | mg |
| starch | 2 | mg |
| talc | 1 | mg |
| stearic acid | 0.5 | mg |

### Biological Methods and Data

Because the compounds of the present invention are active as inhibitors of hYAK3 they exhibit therapeutic utility in treating diseases associated with hYAK3 activity, including but not limited to, anemia, anemias due to renal insufficiency or to chronic disease, such as autoimmunity, HIV, or cancer, and drug-induced anemias, myelodysplastic syndrome, aplastic anemia and myelosuppression, and cytopenia.

Substrate phosphorylation assays are carried out as follows:

YAK3 Scintillation Proximity Assays Using Ser164 of Myelin Basic Protein as the Phosphoacceptor

The source of Ser164 substrate peptide The biotinylated Ser164, S164A peptide (Biotinyl-LGGRDSRAGS*PMARR-OH), sequence derived from the C-terminus of bovine myelin basic protein (MBP) with Ser162 substituted as Ala162, is purchased from California Peptide Research Inc. (Napa, Calif.), and its purity is determined by HPLC. Phosphorylation occurs at position 164 (marked S* above). The calculated molecular mass of the peptide is 2166 dalton. Solid sample is dissolved at 10 mM in DMSO, aliquoted, and stored at −20° C. until use.

The source of enzyme:

hYAK3: Glutathione-S-Transferase (GST)-hYak3-His6 containing amino acid residues 124-526 of human YAK3 (aa 124-526 of SEQ ID NO 2. in U.S. Pat. No. 6,323,318) is purified from baculovirus expression system in Sf9 cells using Glutathione Sepharose 4B column chromatography followed by Ni-NTA-Agarose column chromatography. Purity greater than 65% typically is achieved. Samples, in 50 mM Tris, 150 mM NaCl, 10% glycerol, 0.1% Triton, 250 mM imidazole, 10 mM β-mercapto ethanol, pH 8.0. are stored at −80° C. until use.

Kinase assay of purified hYAK3: Assays are performed in 96 well (Costar, Catalog No. 3789) or 384 well plates (Costar, Catalog No. 3705). Reaction (in 20, 25, or 40 μl volume) mix contained in final concentrations 25 mM Hepes buffer, pH 7.4; 10 mM MgCl₂; 10 mM β-mercapto ethanol; 0.0025% Tween-20; 0.001 mM ATP, 0.1 δCi of [δ-33P]ATP; purified hYAK3 (7-14 ng/assay; 4 nM final); and 4 μM Ser164 peptide. Compounds, titrated in DMSO, are evaluated at concentrations ranging from 50 μM to 0.5 nM. Final assay concentrations of DMSO do not exceed 5%, resulting in less than 15% loss of YAK3 activity relative to controls without DMSO. Reactions are incubated for 2 hours at room temperature and are stopped by a 75 ul addition of 0.19 μg Streptavidin Scintillation Proximity beads (Amersham Pharmacia Biotech, Catalog No. RPNQ 0007) in PBS, pH 7.4, 10 mM EDTA, 0.1% Triton X-100, 1 mM ATP. Under the assay conditions defined above, the Kₘ(apparent) for ATP is determined to be 7.2+/−2.4 μM.

The data for compounds dose responses were plotted as % inhibition, calculated with the data reduction formula 100*(1−[U1−C2)/(C1−C2)]), versus concentration of compound, where U is the unknown value, C1 is the average control value obtained for DMSO, and C2 is the average control value obtained for 0.05M EDTA. DATA were fitted tot h curve described by: y=((Vmax*x)/(K+x)) were Vmax is the upper asymptote and K is the IC50. The results for each compound were recorded aspIC50 calculated as follows: pIC50=−Log 10(K).

Utility of the Present Invention

The compounds of Formula I are useful for treating or preventing disease states in which hYAK3 proteins are implicated, especially diseases of the erythroid and hematopoietic systems, including but not limited to, anemias due to renal insufficiency or to chronic disease, such as autoimmunity, HIV, or cancer, and drug-induced anemias, myelodysplastic syndrome, aplastic anemia, myelosuppression, and cytopenia.

The compounds of Formula I are useful in treating diseases of the hematopoietic system, particularly anemias. Such anemias include an anemia selected from the group comprising: aplastic anemia and myelodysplastic syndrome. Such anemias also include those wherein the anemia is a consequence of a primary disease selected from the group consisting of: cancer, leukemia and lymphoma. Such anemias also include those wherein the anemia is a consequence of a primary disease selected from the group consisting of: renal disease, failure or damage. Such anemias include those wherein the anemia is a consequence of chemotherapy or radiation therapy, in particular wherein the chemotherapy is chemotherapy for cancer or AZT treatment for HIV infection. Such anemias include those wherein the anemia is a consequence of a bone marrow transplant or a stem cell transplant. Such anemias also include anemia of newborn infants. Such anemias also include those which are a consequence of viral, fungal, microbial or parasitic infection.

The compounds of Formula I are also useful for enhancing normal red blood cell numbers. Such enhancement is desirable for a variety of purposes, especially medical purposes such as preparation of a patient for transfusion and preparation of a patient for surgery.

What is claimed is:

1. A method of inhibiting hYAK3 in a mammal; comprising administering to the mammal a therapeutically effective amount of a compound of the Formula I, 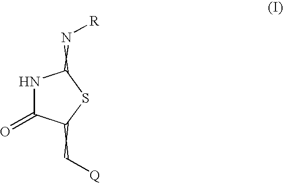
in which
R is selected from: aryl and substituted aryl; and
Q is a substituent of formula (III) 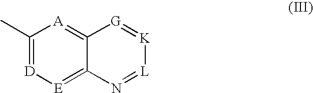
wherein
A, D and E are each independently selected from CR²⁰ and N, and G, K and L are each independently selected from CR²⁰ and N, G and K can optionally form a five-membered ring containing 1-4 nitrogens,
where each R²⁰ is independently selected from the group consisting of: hydrogen, amino, alkylamino, substituted alkylamino, dialkylamino, substituted dialkylamino, hydroxy, alkylaminoalkyl, dialkylaminoalkyl, alkoxy, alkyl, substituted alkyl, aryl, substituted aryl, arylamino, substituted arylamino, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected from hydrogen, C₁-C₄alkyl, aryl and
trifluoromethyl, and R¹¹ and R¹² are independently selected from hydrogen, C₁-C₄alkyl, aryl and trifluoromethyl;
and/or a pharmaceutically acceptable salt, hydrate, solvate, or pro-drug thereof;
provided that not each of G, K and L are N,
further provided that when G is CR²⁰, at least one of A, D, E, K, and L is N,
further provided that when G is N, Q must contain at least three nitrogens.

2. A method of claim 1
in which
R is selected from: aryl and substituted aryl; and
Q is a substituent of formula (III) 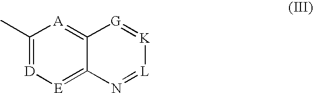
wherein
A, D and E are each independently selected from CR²⁰ and N, and
G, K and L are each independently selected from CR²⁰ and N,
where each R²⁰ is independently selected from the group consisting of: hydrogen, amino, alkylamino, substituted alkylamino, dialkylamino, substituted dialkylamino, hydroxy, alkylaminoalkyl, dialkylaminoalkyl, alkoxy, alkyl, substituted alkyl, aryl, substituted aryl, arylamino, substituted arylamino, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected from hydrogen, C₁-C₄alkyl, aryl and
trifluoromethyl, and R¹¹ and R¹² are independently selected from hydrogen, C₁-C₄alkyl, aryl and trifluoromethyl;
and/or a pharmaceutically acceptable salt, hydrate, solvate, or pro-drug thereof;
provided that not each of G, K and L are N,
further provided that when G is CR²⁰, at least one of A, D, E, K, and L is N,
further provided that at least one of A, D, E, K, and L is N.

3. A compound of formula (V) 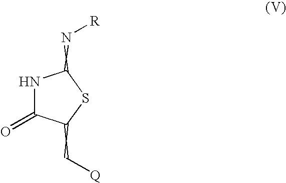
wherein R is C₁-C₁₂aryl or substituted C₁-C₁₂aryl,
Q is a selected from a group consisting of: formula VI, VII, VIII 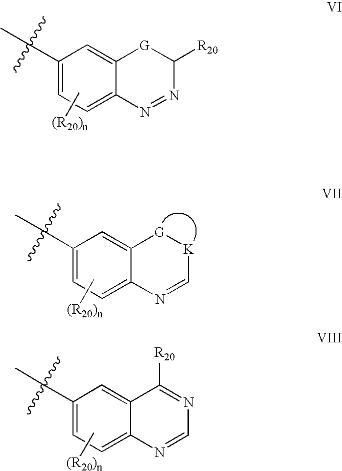
where n is 0-3, G and K are each independently selected from N or CR²⁰,
G and K optionally form a five-membered ring containing 1-4 nitrogens, each R²⁰ is independently selected from the group consisting of: hydrogen, amino, alkylamino, substituted alkylamino, dialkylamino, substituted dialkylamino, alkylaminoalkyl, dialkylaminoalkyl, alkyl, substituted alkyl, aryl, substituted aryl, arylamino, substituted arylamino, halogen, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected from hydrogen, C₁-C₄alkyl, aryl and
trifluoromethyl, and R¹¹ and R¹² are each independently selected from hydrogen, C₁-C₄alkyl, aryl and trifluoromethyl;
and/or a pharmaceutically acceptable salt, hydrate, solvate or pro-drug thereof;
provided that when Q is formula VIII, R²⁰ is not a hydrogen.

4. A compound of claim 3 wherein Q is formula VI; and/or a pharmaceutically acceptable salt, hydrate, solvate or pro-drug thereof.

5. A compound of claim 3 wherein Q is formula VII; and/or a pharmaceutically acceptable salt, hydrate, solvate or pro-drug thereof.

6. A compound of claim 3 wherein Q is formula VIII, and R²⁰ is selected from a group consisting of: amino, alkylamino, dialkylamino, substituted alkylamino, arylamino, oxo, and substituted arylamino; and/or a pharmaceutically acceptable salt, hydrate, solvate or pro-drug thereof.

7. A method of inhibiting hYAK3 in a mammal; comprising administering to the mammal a therapeutically effective amount of a compound of claim 3; and/or a pharmaceutically acceptable salt, hydrate, solvate, or pro-drug thereof.

8. A compound of claim 3 selected from:
(5Z)-2-[(2,6-Dichlorophenyl)amino]-5-(6-quinazolinylmethylidene)-1,3-thiazol-4(5H)-one;
(5Z)-2-[(2,6-Dichlorophenyl)amino]-5-{[4-(4-morpholinyl)-6-quinazolinyl]methylidene}-1,3-thiazol-4(5H)-one;
(5Z)-5-(6-Cinnolinylmethylidene)-2-[(2,6-dichlorophenyl)amino]-1,3-thiazol-4(5H)-one;
(5Z)-2-[(2,6-Dichlorophenyl)amino]-5-(imidazo[1,2-a]quinoxalin-8-ylmethylidene)-1,3-thiazol-4(5H)-one;
N-(4-Chloro-3-{[(5Z)-5-(imidazo[1,2-a]quinoxalin-8-ylmethylidene)-4-oxo-4,5-dihydro-1,3-thiazol-2-yl]amino}phenyl)cyclobutanecarboxamide;
(5Z)-2-[(2,6-Dichlorophenyl)amino]-5-(tetrazolo[1,5-a]quinoxalin-8-ylmethylidene)-1,3-thiazol-4(5H)-one;
(5Z)-5-[(4-Amino-6-quinazolinyl)methylidene]-2-[(2,6-dichlorophenyl)amino]-1,3-thiazol-4(5H)-one trifluoroacetate;
(5Z)-2-[(2,6-Dichlorophenyl)amino]-5-{[4-(methylamino)-6-quinazolinyl]methylidene}-1,3-thiazol-4(5H)-one trifluoroacetate;
6-{(Z)-[2-[(2,6-Dichlorophenyl)amino]-4-oxo-1,3-thiazol-5(4H)-ylidene]methyl}-4(1H)-quinazolinone, piperidine salt;
(5Z)-2-[(2,6-Dichlorophenyl)amino]-5-{[4-(dimethylamino)-6-quinazolinyl]methylidene}-1,3-thiazol-4(5H)-one;
(5Z)-5-{[4-(Methylamino)-6-quinazolinyl]methylidene}-2-[(2,4,6-trichlorophenyl) amino]-1,3-thiazol-4(5H)-one hydrochloride;
4-[(6-{(Z)-[2-[(2,6-Dichlorophenyl)amino]-4-oxo-1,3-thiazol-5(4H)-ylidene]methyl}-4-quinazolinyl)amino]-N,N-dimethylbenzenesulfonamide;
and Ethyl 6-{(Z)-[2-[(2,6-dichlorophenyl)amino]-4-oxo-1,3-thiazol-5(4H)-ylidene]methyl}-1,2,4-benzotriazine-3-carboxylate.

9. A pharmaceutically acceptable salt, hydrate, solvate or pro-drug of a compound of claim 8.

10. A pharmaceutical composition comprising a compound according to claim 3 or a pharmaceutically acceptable salt, hydrate, solvate or pro-drug thereof, and a pharmaceutically acceptable carrier.

11. A process for preparing a pharmaceutical composition containing a pharmaceutically acceptable carrier or diluent and an effective amount of a compound of Formula (V) as described in claim 3 or a pharmaceutically acceptable salt, hydrate, solvate or pro-drug thereof, which process comprises bringing the compound of Formula (V) or a pharmaceutically acceptable salt, hydrate, solvate or pro-drug thereof into association with a pharmaceutically acceptable carrier or diluent.

12. A method of treating or preventing diseases of the erythroid and hematopoietic systems, caused by the hYAK3 imbalance or inappropriate activity; comprising administering to a mammal a therapeutically effective amount of a compound of claim 3, or a pharmaceutically acceptable salt, hydrate, solvate or pro-drug thereof and one or more of pharmaceutically acceptable carriers, diluents and excipients.

13. A method of treating or preventing diseases selected from the group consisting of: anemia, aplastic anemia, myelodysplastic syndrome, myelosuppression, and cytopenia; comprising, administering to a mammal a therapeutically effective amount of a compound of claim 1, or a pharmaceutically acceptable salt, hydrate, solvate or pro-drug thereof and one or more of pharmaceutically acceptable carriers, diluents and excipients.

14. A method of claim 12 in which diseases of the erythroid and hematopoietic systems are selected from the group consisting of: anemia, aplastic anemia, myelodysplastic syndrome, myelosuppression, and cytopenia.

15. The method of claim 12 wherein the mammal is a human.

16. A method of treating diseases of the hematopoietic system, in a mammal in need thereof, which comprises: administering to such mammal a therapeutically effective amount of
a) a compound of Formula (V), as described in claim 3 and/or a pharmaceutically acceptable salt, hydrate, solvate or pro-drug thereof; and
b) EPO or a derivative thereof.
